(19) United States
(12) Patent Application Publication
(10) Pub. No.: US 2017/0202714 A1
(43) Pub. Date: Jul. 20, 2017

(54) Human wearable and detachable incontinence device

(71) Applicants: Darryl Mark Hurwitz, North Palm Beach, FL (US); Judy Daniels, Delray Beach, FL (US); Fred Sandberg, West Palm Beach, FL (US)

(72) Inventors: Darryl Mark Hurwitz, North Palm Beach, FL (US); Judy Daniels, Delray Beach, FL (US); Fred Sandberg, West Palm Beach, FL (US)

(21) Appl. No.: 14/998,518

(22) Filed: Jan. 14, 2016

(51) Int. Cl. A61F 13/505; A61F 13/475; A61F 13/56; A61F 13/62; A61L 15/22; A61F 13/474; A61F 13/534; A61L 15/42; A61L 15/24; A61F 13/476; A61F 13/537

(52) U.S. Cl. CPC A61F 13/505; A61F 13/476; A61F 13/475; A61F 13/5611; A61F 13/62; A61F 13/537; A61F 13/53752; A61F 13/53704; A61F 13/53708; A61F 13/474; A61F 13/53409; A61L 15/42; A61L 15/24; A61L 15/225; A61F 2013/15487

(57) ABSTRACT

A detachable urine flow collection device that integrates with a disposable nonwoven or underwear garment worn by a human male or female. The incontinence device fits through an opening in the disposable nonwoven or underwear garment. The incontinence device is ellipsoid in shape and will be in direct and intimate contact with the users' genitals. This design allows the incontinence device to collect urine when the user urinates in different body positions. The main purpose of the incontinence devise is to channel this urine and liquid away from contact with the user and their skin as the constant contact of the liquid and urine is harmful. The channeling of urine and liquid away from the users skin is accomplished by the design of the incontinence device and the combinations of nonwoven materials that direct, move and store the urine and liquid in the device.

### RELATED APPLICATIONS

The present application is a continuation application of U.S. provisional patent application, Ser. No. 62/103,280, filed Jan. 14, 2015, for HUMAN WEARABLE AND DETACHABLE INCONTINENCE DEVICE, by Darryl Hurwitz, Judy Daniels, Fred sandberg, included by reference herein and for which benefit of the priority date is hereby claimed.

### FIELD OF THE INVENTION

The present invention relates to a human urine incontinence device and, more particularly, to a human urine incontinence device that can be used with regular undergarments and other incontinence devices and that is detachable.

### BACKGROUND OF THE INVENTION

Urinary incontinence is the unintentional loss of urine. More clearly defined as the Inability to hold urine in the bladder due to loss of voluntary control over the urinary sphincter musculature resulting in the involuntary passage of urine. Urinary Incontinence affects 200 million people worldwide. One-third of men and women ages 30-70 have experienced loss of bladder control at some point in their adult lives and may be still living with the symptoms. Although the treatment of urinary incontinence has improved with the advent of new drugs, surgical advancement and management protocols, the standard for managing the involuntary flow of urine, still seems to be fall upon disposable underwear and incontinence devices to allow the user some semblance of hygiene and respectability as they integrate into society. Urinary incontinence is a very expensive medical problem with poor outcomes. Even if the cause and primary symptoms can be improved and managed in the majority of cases, there may still be the occurrence of an involuntary passage of urine. Therefor necessitating the use of a form of incontinence device to absorb this urine. Inadvertently accidents do occur necessitating the laundering of clothes and bedlinens. A social stigma and increased economic cost is also borne by the incontinence sufferer in order to afford the use of these incontinence devices for daily use. Medical issues as a result of being exposed to urine an acidic compound as well as its other components can have a significant affect on the patient skin integrity and health.

Incontinence devices bear many different forms from disposable diapers and pads to briefs/underwear to external collection devices and even indwelling systems (Foley catheter), Davidson U.S. Pat. No. 3,434,869 (1), involving the placement of tubes directly into the bladder. The prior art also describing a disposable diaper, namely a Japanese Patent Application Disclosure No. 1993-277149 (2) describes a disposable diaper comprising a liquid-pervious topsheet, a liquid-impervious backsheet, a liquid-absorbent core disposed between these two sheets and a liquid-resistant upper sheet annularly, extending above the outer surface of the topsheet. In Strickland Et al U.S. Pat. No. 4,253,461 A (3) a disposable absorbent brief is described for use with an adult user, as well as the formation of a thigh seal is proposed. This item is a full brief diaper for use with the adult populations.

An incontinence pad is described in the prior art in Miller US 20060178652 Al (4), namely An absorbent, multi-layer, moisture management pad is provided for wear in the crotch area of apparel. The pad is described, but the invention deals more directly with the textile manufacture. Multiple layers of materials with different properties are used in these pads to draw urine and liquid away from the user and also to be disposable. In Sukinniek Et al U.S. Pat. No. 4,908,026 (5) a Flow distribution system for absorbent pads is discussed, namely in sanitary napkins used for absorption of human exudate. A flow zone control layer which facilitates dispersion of body fluid along the length of the article is described. The use of specific nonwoven materials is used in pads to achieve the channeling of urine and liquid away from the user.

External collection devices such as the Texas condom are found in the prior art Beach U.S. Pat. No. 3,364,932 (6) and are described. In Eaton U.S. Pat. No. 4,769,020 A (7), A male incontinence device in the form of a condom having a urine outlet at its lower end, and a self-adhesive strip is made in one piece that, is attached to, the condom is described. This is an example of a texas catheter utilizing a adhesive strip for adherence of the device to the male penis. The discussion of direct adhesives inside the condom is discussed in Schnieder U.S. Pat. No. 4,581,026 A (8), An external catheter, and using such a catheter, for a male urinary incontinence collection system, as well as such system also including an adhesive attachment for holding the catheter in place is outlined. The adhesive attaching takes the form of a pressure sensitive adhesive coating along the inner surface of the sheath's cylindrical portion, directly behind the sheath's tapered neck section which adheres directly to the penis. In some cases behind the glans or along the shaft of the penis. As regards External collection devices that can be used for females these are severely limited by anatomy as well as the environment of the female anatomy, in Kay Et al, US 20110028922 A1 (9), a flange inserted into the vagina is described to hold the device over the urinary meattus. In MacDougall U.S. Pat. No. 4,822,347 A (10), a tubular sheath integral with a funnel and a urine-conducting pipe is secured to a pad of adhesive material pad that is dimensioned so that it can be stuck to the skin of the wearer in the region immediately surrounding the urethral orifice (meatus). Another form of female external catheter is described in Blackmon U.S. Pat. No. 4,484,917 A (11), were the device is described as an adhesive surface, a pliable but resilient stabilizer, and a main body and an outlet tube. This external catheter covers the female genitali in entirety to funnel away urine and vaginal discharge and is attached by an adhesive.

Indwelling systems increase the element of infection due to a foreign body being introduced into the body. Mechanical damage to the body structure is also a possibility, as a result of physical stresses placed on the bodies structures by these indwelling systems, such as bladder and urethral rupture.

External collection devices are often exclusively found in use with the male population. The most common external collection device used for male patients is the external condom catheter, comprising a tube connected to the end of a condom. The condom collection unit that is attached to the penis with an adhesive or pressure band (i.e. commonly referred to as a “Texas catheter”). These External condoms are prone to detachment and urine loss. These devices are generally ineffective and uncomfortable for the wearer. External condom devices used in an active population of patients are unreliable to allow its use for multiple hours without falling off, or becoming increasingly uncomfortable. In one version the external condom catheter is held in place on the penis by an elastic belt that fits around the patient's waist and/or legs. Force exerted by the belt holds the condom (collection unit) in place. Another variation of the condom catheter utilizes an adhesive unit that fits around only a tip of the penis (the glans), another is a variant were the adhesive and condom is applied to the shaft of the penis when the penis is erect, thus encasing the entire penis when flaccid. The external condom catheter is directly connected to a collection bag and tubing to direct the fluid away from the patient. The penis constantly changes size during the day and the condom collection assembly is prone to detachment. A dry genital area cannot be maintained when the condom is displaced from its initial position and therefore leaks urine. Adhesives used in the attachment of the external condom catheter have the potential of skin breakdown due to tissue movement, an acidic environment as well as long term tissue exposure to adhesive chemicals and constituents. Another urine collection approach comprises a custom brief and a condom-like catheter within a pocket formed in the brief. The urine exits the condom catheter into a collection bag. These units are expensive and, like the other condom-type units described above, fails when the penis contracts. The use of external collection devices in females is even more problematic, anatomically finding a method of attachment to the female genitalia has been difficult, and the use of adhesives and glues has been virtually impossible due to anatomical constraints as well as a damp environment in close proximity to the device.

Disposable diapers and briefs/underwear come in many forms from full garments to liners and pads. The determination of use of these items is determined by the amount of urine flow expected, as well as other excretory functions (fecal), the ability of the user to change the devise, and the potential of use of these items with traditional underwear. Pads and liners have a limited absorbency rate and may be ideal for patients that have very small amounts of urine flow or leakage. Full garments are used for patients that have high output levels and or the possibility of fecal incontinence. The patient who fits in the middle of these two extremes really has no choice. Use a full garment or risk an incontinence episode due to urine outflow excessive of the device. All of these devices rely on absorption systems for urine. Even with the system working; urine and a highly acidic environment are still in proximity with the user's skin leading to skin breakdown. A patient using a full garment has the difficulty of changing and replacing the garment to avoid these issues. This can mean changing out of one's clothes or disassembling the garment to allow application. The problems with the known and available prior art devices, and well documented in medical literature, is the psychologically debilitating effect of incontinent garment use; and also the environmental considerations resulting from the disposal of the vast variety and array of different types of incontinence devices and nonwoven materials. Nonwoven materials are extremely difficult for the environment as they are not biodegradable and easy to breakdown.

### SUMMARY OF THE INVENTION

In accordance with the present invention, there is provided a detachable urine flow collection device that integrates with a disposable nonwoven or underwear garment worn by a human male or female. The incontinence device fits through an opening in the disposable nonwoven or underwear garment. The incontinence device is ellipsoid in shape and will be in direct and intimate contact with the users' genitals. This design allows the incontinence device to collect urine when the user urinates in different body positions. The main purpose of the incontinence devise is to channel this urine and liquid away from contact with the user and their skin as the constant contact of the liquid and urine is harmful. The channeling of urine and liquid away from the users skin is accomplished by the design of the incontinence device and the combinations of nonwoven materials that direct, move and store the urine and liquid in the device.

It would be advantageous to provide a detachable urine flow collection device that can function with a disposable nonwoven or underwear garment.

It would be advantageous to provide a detachable urine flow collection device that can function with a disposable nonwoven or underwear garment.

It would be advantageous to provide a detachable urine flow collection device that by functioning with a disposable nonwoven or underwear garment will allow only the detachable urine flow collection device to be soiled with urine and liquid and changed

It would be advantageous to provide a detachable urine flow collection device that moves the liquid and urine away from the users' skin.

It would be advantageous to provide a detachable urine flow collection device that directs and drives and channels the urine and liquid away from the users' skin.

It would be advantageous to provide a detachable urine flow collection device that can be utilized with the user being in multiple body positions.

It would be advantageous to provide a detachable urine flow collection device that will allow the male user with the diagnosis of retro penis to use a detachable urine flow collection device.

It would be advantageous to provide a detachable urine flow collection device that can be changed without removing the users' cloths.

It would be advantageous to provide a detachable urine flow collection device that can be changed without removing the users' disposable nonwoven or underwear garment.

It would be advantageous to provide a detachable urine flow collection device that can be removed and applied with the user in multiple body positions.

It would be advantageous to provide a detachable urine flow collection device that can accommodates all levels of urine capacity.

It would be advantageous to provide a detachable urine flow collection device that will be smaller and lower the environmental impact.

It would be advantageous to provide a detachable urine flow collection device that will be less bulky under the users' clothes.

It would be advantageous to provide a detachable urine flow collection device that is effective and comfortable to wear.

It would be advantageous to provide a detachable urine flow collection device which is non-irritating to the skin, and which does not use adhesives, tape, glue or other constricting elements to attached directly to the genitalia.

It would be advantageous to provide a detachable urine flow collection device which is especially designed for active users.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a front view of a this front view demonstrates the relationship between the detachable urine flow collection device as it relates to its use and connection with the front aspect of a disposable nonwoven or underwear garment;

FIG. 2 is a left exploded view of a this exploded upper perspective view shows the different parts of the detachable urine flow collection device and how they relate with one another. the genital ring and opening area has an ellipsoid opening and is made of a soft moldable nonwoven material. the genital ring and opening area joins to the closure ring. the next level of the detachable urine flow collection device moving from the top to the bottom is the quick wicking area and below the quick wicking area is the urine and liquid absorbing area;

FIG. 3 is a top exploded view of a this exploded upper perspective view demonstrates the different components of the detachable urine flow collection device in relation to a disposable nonwoven or underwear garment as well as demonstrating the relationship between the different parts of the detachable urine flow collection device. the detachable urine flow collection device fits through the garment opening. the genital ring and opening area is represented on the inside anterior surface of the disposable nonwoven or underwear garment. the tab attaches to the front and top of the genital ring and opening area. the genital ring and opening area connects directly to the closure ring which is also inside the disposable nonwoven or underwear garment. as the detachable urine flow collection device passes through the garment opening the next level of the detachable urine flow collection device is the quick wicking area which then attaches to the urine and liquid absorbing area. the quick wicking area and urine and liquid absorbing area of the detachable urine flow collection device are outside the disposable nonwoven or underwear garment;

FIG. 4 is a left detail view of a this left detail view demonstrates the detachable urine flow collection device. the tab is shown attached to the front top surface of the genital ring and opening area. the genital ring and opening area is attached to the closure ring. the inferior surface of the closure ring is comprised of velcro tape or stick type fasteners. these velcro tape or stick type fasteners will adhere to the opposite type material around the garment opening on the inside anterior surface of the disposable nonwoven or underwear garment; and

FIG. 5 is a this cross sectional view demonstrates the non-woven layer configuration of the detachable urine flow collection device. all three areas of the detachable urine flow collection device are shown from top to bottom.

For purposes of clarity and brevity, like elements and components will bear the same designations and numbering throughout the Figures.

### DESCRIPTION OF THE PREFERRED EMBODIMENT

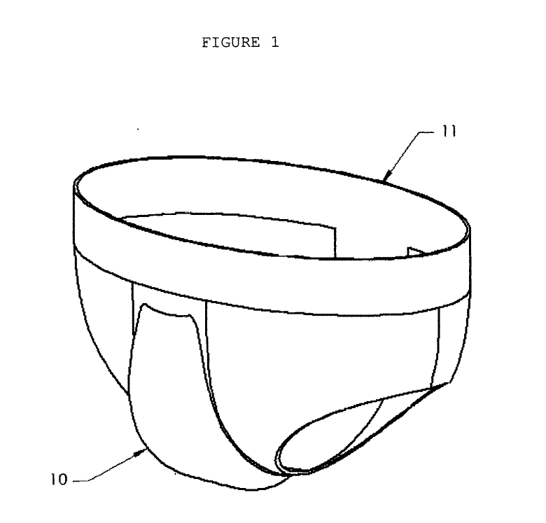

FIG. 1 is a front view that demonstrates the relationship between the detachable urine flow collection device 10 as it relates to its use with a disposable nonwoven or underwear garment 11.

The detachable urine flow collection device 10 is designed to fit through the garment opening 12 in the front portion of a disposable nonwoven or underwear garment 11. The detachable urine flow collection device 10 can be applied or removed from the disposable nonwoven or underwear garment 11 attachment as needed. The genital ring and opening area 20 of the external collection assembly of the present invention connects with the inner surface of the garment opening 12 formed in a disposable nonwoven or underwear garment 11. The genital ring and opening area 20 is the top ⅓ of the detachable urine flow collection device 10. The connection with the disposable nonwoven or underwear garment 11 is an intimate contact, and waterproof.

An alternative embodiment of the detachable urine flow collection device 10 is structurally the detachable urine flow collection device 10 has another shape to allow a user to wear it with an absorbent disposable nonwoven or underwear garment 11. The front of The detachable urine flow collection device 10 anatomically is toward the front of the user; the opening then progresses toward the back or posterior of the user between the upper part of the lower limbs.

An alternative embodiment to the design of the detachable urine flow collection device 10 allows for the direction and movement of the urine and liquids away from the user, and is obtained by utilizing specific nonwoven materials in layers, and multiple combinations, to achieve the purpose. The combinations of these nonwoven materials are designed to obtain the optimal results as the urine and liquid pass through the different areas of detachable urine flow collection device 10

In operation of the invention the user applies the detachable urine flow collection device 10 to a disposable nonwoven or underwear garment 11. The application takes place by placing the detachable urine flow collection device 10 through the garment opening 12. The genital ring and opening area 20 adheres to the disposable nonwoven or underwear garment 11 by the closure ring 23 This is achieved by Velcro tape type fasteners, or sticks tape type fasteners on the underside of the nonwoven material closure ring 23, that adhere to an opposite material on the disposable nonwoven or underwear garment 11.

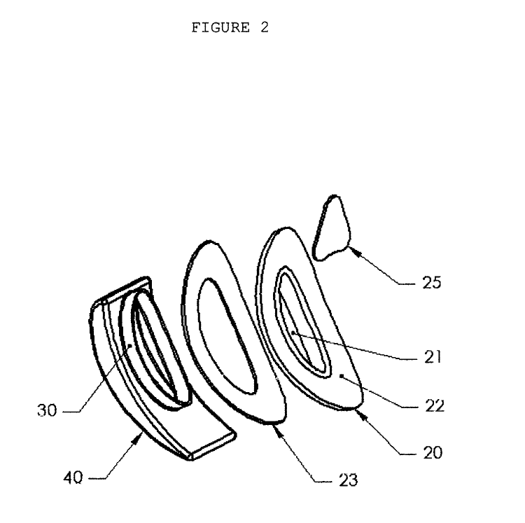

FIG. 2 is an exploded upper perspective view that shows the different parts of the detachable urine flow collection device 10 and how they relate with one another. The genital ring and opening area 20 has an ellipsoid opening and is made of a soft moldable nonwoven material 22. The genital ring and opening area 20 joins to the closure ring 23. The next level of the detachable urine flow collection device 10 moving from the top to the bottom is the quick wicking area 30 and below the quick wicking area 30 is the urine and liquid absorbing area 40.

The detachable urine flow collection device 10 structurally is ellipsoid in shape. The front of the detachable urine flow collection device 10 is the widest part of the ellipse, and moving backward it ellipses backward to its narrowest part. The detachable urine flow collection device 10 maintains this ellipsoid shape throughout its height. The detachable urine flow collection device 10 consists of three areas. The genital ring and opening area 20, a quick wicking area 30, and a urine and liquid absorbing area 40. The length of the genital ring and opening area 20, is shorter than that of the other two areas.

An alternative embodiment to the design of the genital ring and opening area 20 is that the front of the genital ring and opening area 20 is semi-circular in shape and moving backward it ellipses backward to its narrowest part.

A second alternative embodiment to the design of the front of the genital ring and opening area 20 is Semi-circular in shape, and moving backward is rectangular in shape. The back of the genital ring and opening area 20 is also semi-Circular in shape.

A third alternative embodiment to the design, of the genital ring and opening area 20 structurally that makes up the top ⅓ of the detachable urine flow collection device 10 is that it has a alternatively shaped Opening, made of a soft moldable nonwoven material 22. The shape of the opening will be the best fit for the most intimate contact, with the user's genitalia. The shape of the opening will be the best shape fit to form the most intimate seal between the male and female genitals. The material of the opening can be molded around the genitalia of the user upon application.

An alternative embodiment to the design of the closure ring 23 is a nonwoven material that attaches to the top edge of the ellipsoid opening, at ninety degrees to the body of the detachable urine flow collection device 10. The underside of the nonwoven Closure Ring 23 (23) material is comprised of Velcro Tape or Stick Type Fasteners 24 (24).

A second alternative embodiment to the design of the closure ring 23 is a nonwoven material that attaches to the top edge of the ellipsoid opening, at ninety degrees to the body of the detachable urine flow collection device 10. The underside of the nonwoven closure ring 23 material is comprised of the newer forms of elasticized nonwoven Velcro tapes or other emerging in the absorbent garment (diaper) brief market, that are designed to form an intimate close fit between two nonwoven surfaces and may also be waterproof.

A third alternative embodiment to the closure ring 23 is a nonwoven material that attaches to the top edge of the ellipsoid opening, at ninety degrees to the body of the detachable urine flow collection device 10. The top side of the nonwoven closure ring 23 material is comprised of Velcro Tape or Stick Type Fasteners 24 (24), or can also be one of the newer forms of elasticized nonwoven Velcro tapes emerging in the absorbent garment (diaper) brief market.

An alternative embodiment to the design of the genital ring and opening area 20 involves multiple tab 25 or loose flaps of nonwoven material will be attached to the font and top surface of the ellipsoid opening.

In operation of the invention, the function of the detachable urine flow collection device 10 is to direct urine and liquids away from the user to a urine and liquid absorbing area 40 of the device for storage. The direction and movement of the urine and liquids away from the user, is obtained by utilizing specific nonwoven materials in layers, and combinations, to achieve the purpose.

The nonwoven materials possess various properties of a hydrophobic and a hydrophilic nature. Hydrophobic is water molecule repelling and hydrophilic is water molecule attracting. These specific properties of the nonwoven materials and the combinations allow the movement, directing, and capturing and storage of urine and liquids to take place in the detachable urine flow collection device 10. The layers of the three distinctive areas of the detachable Urine Flow Device are sometimes duplicated from the area above.

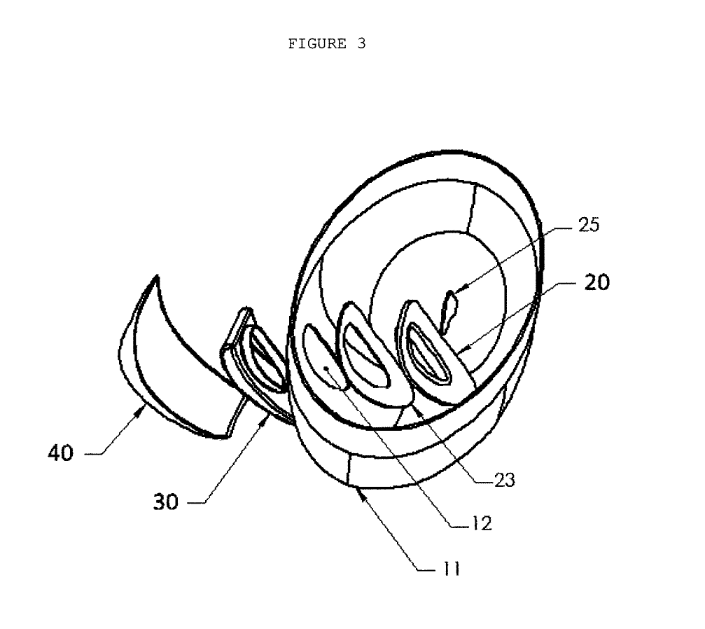

FIG. 3 is an exploded upper perspective view demonstrates the different components of the detachable urine flow collection device 10 in relation to a disposable nonwoven or underwear garment 11 as well as demonstrating the relationship between the different parts of the detachable urine flow collection device 10.

The detachable urine flow collection device 10 fits through the garment opening 12. The genital ring and opening area 20 is represented on the inside anterior surface of the disposable nonwoven or underwear garment 11. The genital ring and opening area 20 structurally makes up the top ⅓ of the detachable urine flow collection device 10 and has an ellipsoid opening, made of a soft moldable nonwoven material 22. The tab 25 attaches to the front and top of the genital ring and opening area 20. The genital ring and opening area 20 connects directly to the closure ring 23 which is also inside the disposable nonwoven or underwear garment 11.

When the detachable urine flow collection device 10 passes through the garment opening 12 and is in place the genital ring and opening area 20 and the closure ring 23 are inside the disposable nonwoven or underwear garment 11 and the quick wicking area 30 and urine and liquid absorbing area 40 are outside the disposable nonwoven or underwear garment 11.

Below the genital ring and opening area 20 is the quick wicking area 30, the middle ⅓ area of the detachable urine flow collection device 10. The specific purpose of the quick wicking area 30 is to move the urine and liquid as quickly as possible to the urine and liquid absorbing area 40. The quick wicking area 30 or Middle ⅓ area of the detachable urine flow collection device 10 is also ellipsoid in shape and is longer from back to front than the genital ring and opening area 20 of the detachable urine flow collection device 10.

In operation of the invention, the quick wicking area 30 is also made of layers of nonwoven material that are designed to channel urine and liquid down and away from the user and the genital ring and opening area 20. These layers' act in a specific fashion in order to wick the urine and liquid down and away from the user; as well as to direct the urine and liquid to a specific portion of the detachable urine flow collection device 10, namely the urine and liquid absorbing area 40.

The urine and liquid absorbing area 40, bottom ⅓ area is also ellipsoid in shape and is approximately the same size as the quick wicking area 30 or middle ⅓ area in length from front to back.

In operation of the invention, the urine and liquid absorbing area 40 is also made of layers of nonwoven material that are designed to channel urine and liquid down and away from the user and the quick wicking area 30. These layers' act in a specific fashion in order to wick the urine and liquid down and away from the user; as well as to direct the urine and liquid to a specific portion of the detachable urine flow collection device 10, namely the core of the urine and liquid absorbing area 40.

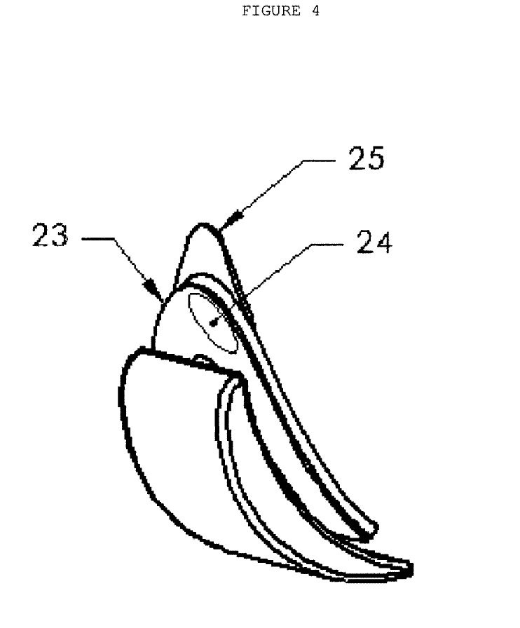

FIG. 4 is a left detail view demonstrates the detachable urine flow collection device 10. The tab 25 is shown attached to the front top surface of the genital ring and opening area 20. The genital ring and opening area 20 is attached to the closure ring 23. The inferior surface of the closure ring 23 is comprised of velcro tape or stick type fasteners 24. These velcro tape or stick type fasteners 24 will adhere to the opposite type material around the garment opening 12 on the inside anterior surface of the disposable nonwoven or underwear garment 11.

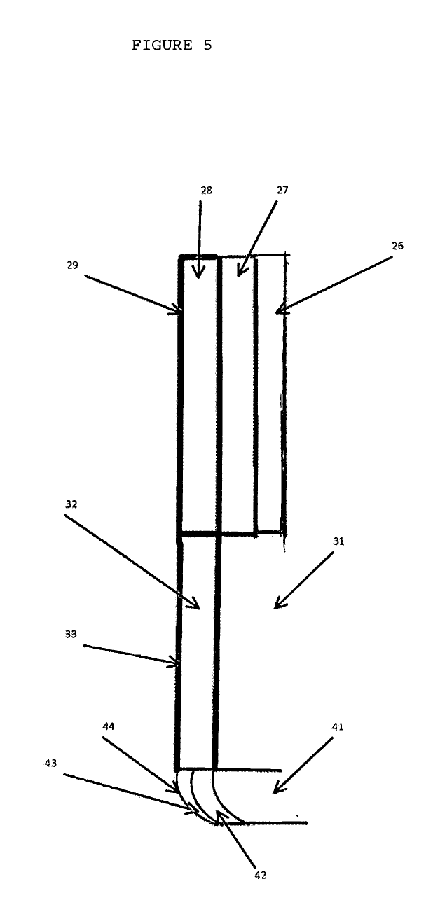

FIG. 5 is a cross sectional view demonstrates the nonwoven layer configuration of the detachable urine flow collection device 10. All three areas of the detachable urine flow collection device 10 are shown from top to bottom. The top 1/3, genital ring and opening area 20, the middle 1/3, quick wicking area 30, and the bottom 1/3, urine and liquid absorbing area 40 are shown in cross section. The cross section also demonstrates how the nonwoven material layers are continuous as you move from the top of the detachable urine flow collection device 10 to the bottom.

The Genital Ring and opening Area 20 is made up of the following nonwoven layers, and is described moving from the innermost layer closest to the open chamber of the detachable urine flow collection device 10 moving outward. The innermost first layer is gr layer 1 (through air bond (tab) Nonwoven) 26 that provides separation from any wet area and the skin. The second layer is gr layer 2 (hydrophobic nonwoven material) 27 The third layer is gr layer 3 (hydrophilic nonwoven material) 28. The fourth and final layer is the gr layer 4 (polyethylene nonwoven material) 29, that is the outside layer of the detachable urine flow collection device 10, and is entirely hydrophobic or waterproof.

An alternative embodiment of the nonwoven layers of the genital ring and opening area 20, is that the genital ring and opening area 20 alternatively, is made up of the following nonwoven layers, and is described moving from the innermost layer closest to the open chamber of the genital ring and opening area 20 moving outward. The combination of nonwoven layers are sequenced to best accommodate the function of the genital ring and opening area 20, namely keeping the genital areas dry and channeling the urine and liquid away from this area and the user toward the quick wicking area 30 in the fastest possible time.

The quick wicking area 30, Includes the following nonwoven layers, and is described moving from the innermost layer closest to the open chamber of the detachable urine flow collection device 10 moving outward. The innermost first layer is a continuation of the first layer gr layer 1 (through air bond (tab) nonwoven) 26 of the genital ring and opening area 20 and is the qw layer 1 (through air bond (tab) nonwoven) 31 that provides separation from the wet area and the skin. The second layer is a continuation of the gr layer 3 (hydrophilic nonwoven material) 28 from the genital ring and opening area 20. This qw layer 2 (hydrophilic nonwoven material is pleated and angled back toward the back and bottom of the detachable urine flow collection device 10 to facilitate the movement of urine and liquid toward the bottom and back of the urine and liquid absorbing area 40, bottom ⅓ area. The third and final layer is a continuation of the gr layer 4 (polyethylene nonwoven material) 29 of the genital ring and opening area 20, and is the qw layer 3 (polyethylene nonwoven material that is the outside layer of the entire detachable urine flow collection device 10, and is entirely hydrophobic or waterproof.

An alternative embodiment of the nonwoven layers of the quick wicking area 30, alternatively, is that the quick wicking area 30 is made up of the following nonwoven layers, and is described moving from the innermost layer closest to the open chamber of the quick wicking area 30 moving outward. The combination of nonwoven layers are sequenced to best accommodate the function of the quick wicking area 30, namely channeling and moving the urine and liquid away from this area and the user toward the urine and liquid absorbing area 40, in the fastest possible time.

The urine and liquid absorbing area 40, is made up of the following layers, and is described moving from the innermost layer closest of the open chamber of the detachable urine flow collection device 10 moving outward. The innermost first layer is a continuation of the first layer the qw layer 1 (through air bond (tab 25) nonwoven of the quick wicking area 30 and is the ul layer 1 (through air bond (tab 25) nonwoven that provides separation from the wet area and the skin. The second layer the ul layer 2 (hydrophilic nonwoven material) 42 is a continuation of the qw layer 2 (hydrophilic nonwoven material from the quick wicking area 30. This second layer is pleated and angled back toward the back and bottom of detachable urine flow collection device 10 to facilitate the movement of urine and liquid toward the bottom of urine and liquid absorbing area 40. The third layer is the ul layer 3 (the core) 43, made up of wood pulp or cellulose which really is the core of detachable urine flow collection device 10 to absorb the urine and liquids. This provides the absorbing capacity of the detachable urine flow collection device 10. A Super absorbent polymer-Sodium Polyacrylate, (SAP) is interwoven throughout the cellulose to allow ul layer 3 (the core) 43 to absorb urine and more liquid and maintain control of this liquid when the core is under physical pressure. The liberal use of SAP in the ul layer 3 (the core) 43, allows less wood pulp or cellulose to be used thus creating a more streamlined garment. The Fourth and final

An alternative embodiment of the nonwoven layers of the quick wicking area 30, alternatively, is that urine and liquid absorbing area 40 is made up of the following nonwoven layers, and is described moving from the innermost layer closest to the open chamber of the urine and liquid absorbing area 40 moving outward. The combination of nonwoven layers are sequenced to best accommodate the function of the urine and liquid absorbing area 40, namely to direct the urine and liquid to ul layer 3 (the core) 43 as well as absorb the urine and liquid, in the fastest possible time, and maintain control of this urine and liquid when the urine and liquid absorbing area 40 is under pressure.

In operation of the invention, the user applies the detachable urine flow collection device 10 to a disposable nonwoven or underwear garment 11. The application takes place by placing the detachable urine flow collection device 10 through the garment opening 12. The genital ring and opening area 20 adheres to the disposable nonwoven or underwear garment 11 by the closure ring 23 and the adherence methods that have been described,

When the user applies the disposable nonwoven or underwear garment 11, the genital ring and opening area 20 is molded around the genitals of the user. In the case of a male user the genital ring and opening area 20 can be molded around the penis or around the entire male genitalia. In the case of a female user the genital ring and opening area 20 can be molded around the vulva and external genitalia. The soft moldable nonwoven material 22 of the genital ring and opening area 20 allows the user to form a close intimate bond between the detachable urine flow collection device 10 and the user's genitals. Because the detachable urine flow collection device 10 is ellipsoid in shape and is in direct contact with the users genitals, it allows the detachable urine flow collection device 10 to collect urine when the user urinates while in sitting, standing and even when lying down. Urine is a naturally occurring excrement of the human body comprised mainly of water (liquid) and other constituents. The main purpose of the detachable urine flow collection device 10 is to channel this liquid and its other constituents away from contact with the user and their skin as the constant contact of the liquid and urine is harmful. This contact can cause skin breakdown and infection. Normal voiding volumes are 210-300 cc's of urine in a single episode. Urine stream speeds have been recorded as 235 and 325 cm/s. The movement of liquid and urine through the detachable urine flow collection device 10 is governed by the following factors:

Gravity will always draw the liquid and urine downward through the different levels of the device. Gravity is further aided by the properties of the nonwoven materials making up the detachable urine flow collection device 10 to also direct the liquid and urine.

The nonwoven materials can direct liquid and urine one way thus moving the liquid and urine away from the user and providing a dry barrier to the skin. Nonwoven materials can by hydrophobic in nature and actually move the liquid and urine directionally through the device using gravity and the repelling nature of the nonwoven material. Nonwoven materials can be hydrophilic in nature, were the material will actually attract and trap the liquid and urine in its fibers. By using these materials in the specific sequence in the detachable urine flow collection device 10 liquid and urine can be directed, driven, and collected. The overall movement of liquid and urine through the detachable urine flow collection device 10 is downward from the top of the device to the bottom, and backward from the front of the device to the back, and outward from the inner chamber to the outermost layer of the detachable urine flow collection device 10. Because of these factors the entire amount of liquid and urine entering the detachable urine flow collection device 10 will not make contact with the side walls and layers at all the same levels of the detachable urine flow collection device 10. That is why the layering of the different levels of the detachable urine flow collection device 10 is sometimes duplicated.

In operation of the invention a urination episode by the user wearing the detachable urine flow collection device 10 is described. This description described the flow of liquid and urine through the detachable urine flow collection device 10. Because of the intimate contact with the detachable urine flow collection device 10, when the user urinates the urine will pass through the ellipsoid Opening 21 and makes contact with the first layer of the genital ring and opening area 20 the gr layer 1 (through air bond (tab) Nonwoven) 26. The urine once it passes through this layer begins to move outwards to the successive layers of the genital ring and opening area 20, The gr layer 1 (through air bond (tab) Nonwoven) 26 layer is essentially one way and will not allow the urine and liquid to come in contact with the users skin. The urine is affected by the force of gravity and makes contact with gr layer 2 (hydrophobic nonwoven material) 27 layer of the genital ring and opening area 20. This layer is hydrophobic in nature and thus repels and moves the urine and liquid downward and outward toward the gr layer 3 (hydrophilic nonwoven material) 28 of the genital ring and opening area 20. This layer is hydrophilic in nature and will attract the liquid and urine into this layer, some of this liquid and urine will be bound into this layer due to its hydrophilic properties. The rest of the urine will travel down and outward until it comes in contact with the gr layer 4 (polyethylene nonwoven material) 29 this is the outermost layer of the genital ring and opening area 20 and in fact the entire detachable urine flow collection device 10; it is a polyethylene nonwoven and is entirely waterproof.

The liquid and urine will continue by gravity to move downward into the next level of the detachable urine flow collection device 10. Once the liquid enters the quick wicking area 30 it interacts with the different layers of this level. The qw layer 1 (through air bond (tab) nonwoven) 31 is a one-way barrier and allows the liquid to move outward and downward by the force of gravity to the subsequent layers of the quick wicking area 30. By the qw layer 1 (through air bond (tab) nonwoven) 31 layer being a one-way layer this also prevents the liquid and urine from coming in contact with the user. The urine and liquid continues to the next layer of the quick wicking area 30. This layer is the qw layer 2 (hydrophilic nonwoven material) 32) it is hydrophilic in nature and will attract the liquid and urine. The liquid and urine that is not bound into the qw layer 2 (hydrophilic nonwoven material) 32 will continue to move down and outward until they reach the qw layer 3 (polyethylene nonwoven material) 33 this layer is entirely waterproof and will continue to direct the liquid and urine downward to the next level of the detachable urine flow collection device 10. The liquid and urine continues to move downward and outward into the urine and liquid absorbing area 40. As the liquid and urine comes in contact with the first layer the ul layer 1 (through air bond (tab) nonwoven) 41 it moves through this one-way layer to the next layer of the urine and liquid absorbing area 40 the ul layer 2 (hydrophilic nonwoven material) 42 this layer will absorb some of the liquid and urine and direct the remaining liquid and urine to the ul layer 3 (the core) 43. The ul layer 3 (the core) 43 constituents of wood pulp cellulose and SAP will absorb the majority of the remaining liquid and urine. This ul layer 3 (the core) 43 primarily by the inclusion of SAP, also allows the layer to maintain liquid and urine even under physical pressure, such as when parts of the detachable urine flow collection device 10 are compressed by movement and sitting down. This feature allows the user to maintain the detachable urine flow collection device 10 attached even after a urination event has taken place. The user is able to determine when the detachable urine flow collection device 10 can be removed. The design of the detachable urine flow collection device 10 even allows for more than one urination event to take place before replacement. To replace the detachable urine flow collection device 10, the disposable nonwoven or underwear garment 11 is pulled down to above the knees by the user. The user then removes the detachable urine flow collection device 10 by pulling the tab 25, this breaks the closure between the closure ring 23 and the disposable nonwoven or underwear garment 11 and allows the detachable urine flow collection device 10 to be removed and a new one applied. The used detachable urine flow collection device 10 can then be disposed of.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A human wearable and detachable incontinence device for that when worn, will accept and trap urine and keep it away from contact with the user's skin until the device it is detached and disposed of, comprising:
means for a detachable urine flow collection device that can be attached to an absorbent garment (diaper) brief or traditional underwear garment (garment) worn by a human. the detachable urine flow collection device consisting of three areas. a genital ring and opening area, a quick wicking area, and a urine and liquid absorbing area. the purpose of the detachable urine flow collection device is to direct urine and liquids away from the user to a urine and liquid absorbing area of the device. the direction and movement of urine and liquids away from the user, is attained by utilizing specific nonwoven materials in layers, and combinations, to achieve the purpose. the specific properties of the nonwoven materials and the combinations allow this movement and capturing and storage of urine and liquids to take place in the detachable urine flow collection device. the detachable urine flow collection device is designed to fit through an opening in the front portion of an disposable nonwoven garment (diaper) or brief, as well as a traditional underwear garment. the detachable urine flow collection device can be applied or removed from the attachment as needed. the genital ring and opening area of the detachable urine flow collection device assembly of the present invention mates with an opening formed in an absorbent disposable nonwoven or underwear garment. the genital ring and opening area is the top ⅓ of the detachable urine flow collection device. the connection is an intimate contact, and waterproof;
means for the application of the detachable urine flow collection device. the detachable urine flow collection device fits through the garment opening in the front anterior portion of the disposable nonwoven or underwear garment. the detachable urine flow collection device attaches to the disposable nonwoven or underwear garment by velcro tape or stick type fasteners on the underside surface of the closure ring of the urine and liquid absorbing area;
means for the application of the detachable urine flow collection device to the disposable nonwoven or underwear garment. the detachable urine flow collection device fits through the garment opening in the front anterior portion of the disposable nonwoven or underwear garment. the detachable urine flow collection device attaches to the disposable nonwoven or underwear garment by velcro tape or stick type fasteners on the underside surface of the closure ring of the urine and liquid absorbing area. these velcro tape or stick type fasteners on the underside of the closure ring attached directly to the opposite material on the inside surface of the disposable nonwoven or underwear garment opening, structurally connected to said means for the application of the detachable urine flow collection device. the detachable urine flow collection device fits through the garment opening in the front anterior portion of the disposable nonwoven or underwear garment. the detachable urine flow collection device attaches to the disposable nonwoven or underwear garment by velcro tape or stick type fasteners on the underside surface of the closure ring of the urine and liquid absorbing area, and symmetrically connected to said means for a detachable urine flow collection device that can be attached to an absorbent garment (diaper) brief or traditional underwear garment (garment) worn by a human. the detachable urine flow collection device consisting of three areas. a genital ring and opening area, a quick wicking area, and a urine and liquid absorbing area. the purpose of the detachable urine flow collection device is to direct urine and liquids away from the user to a urine and liquid absorbing area of the device. the direction and movement of urine and liquids away from the user, is attained by utilizing specific nonwoven materials in layers, and combinations, to achieve the purpose. the specific properties of the nonwoven materials and the combinations allow this movement and capturing and storage of urine and liquids to take place in the detachable urine flow collection device. the detachable urine flow collection device is designed to fit through an opening in the front portion of an disposable nonwoven garment (diaper) or brief, as well as a traditional underwear garment. the detachable urine flow collection device can be applied or removed from the attachment as needed. the genital ring and opening area of the detachable urine flow collection device assembly of the present invention mates with an opening formed in an absorbent disposable nonwoven or underwear garment. the genital ring and opening area is the top ⅓ of the detachable urine flow collection device. the connection is an intimate contact, and waterproof;
means for the genital ring and opening structurally makes up the top ⅓ of the detachable urine flow collection device and is shaped like an ellipse with the opening, made of a soft moldable nonwoven material. the front of the genital ring is the widest part of the ellipse, and moving backward it ellipses backward to its narrowest part. the closure ring is a nonwoven material that attaches to the top outside edge of the ellipsoid opening, at ninety degrees to the body of the detachable urine flow collection device. the underside of this nonwoven material closure ring is comprised of velcro tape type fasteners, or sticks tape type fasteners, or can also be one of the newer forms of elasticized nonwoven velcro tapes emerging in the absorbent garment (diaper) brief market. the tab a loose flap of nonwoven material will be attached to the font and top of the ellipsoid opening. the genital ring area is made up of the following nonwoven layers, and is described moving from the innermost layer closest to the open chamber of the detachable urine flow collection device moving outward. the innermost first layer is a through air bond (tab) nonwoven that provides separation from any wet area and the skin. the second layer is a hydrophobic nonwoven material. the third layer is a hydrophilic nonwoven material. the fourth and final layer is a polyethylene nonwoven material that is the outside layer of the detachable urine flow collection device, and is entirely hydrophobic or waterproof. the detachable urine flow collection device is designed to fit through an opening in the front portion of an absorbent disposable nonwoven garment (diaper) or brief, as well as a traditional underwear garment (garment). the function of the genital ring and opening is to accommodate the wearers genitals, furthermore the genital ring is the part of the detachable urine flow collection device that accommodates the attachment with the absorbent disposable nonwoven garment through the closure ring. the genital ring and opening of the external collection assembly of the present invention mates with the opening formed in an absorbent disposable nonwoven garment. the connection is an intimate contact, and waterproof. the underside surface of the closure ring, the fastener will make contact with the opposite nonwoven fasteners on the inside surface of the absorbent disposable non-woven garment that lines the opening of the absorbent disposable nonwoven garment. with a user wearing an absorbent disposable nonwoven garment, the widest part of the ellipsoid opening anatomically is toward the front of the user; the opening then ellipses toward the narrowest part, at the back or posterior of the user. the detachable urine flow collection device can be applied or removed from the attachment as needed by pulling on the tab and lifting the detachable urine flow collection device out of the absorbent disposable nonwoven garment. the genital ring or opening top ⅓ of the detachable urine flow collection device then progresses to the quick wicking or middle ⅓ of the detachable urine flow collection device, structurally connected to said means for a detachable urine flow collection device that can be attached to an absorbent garment (diaper) brief or traditional underwear garment (garment) worn by a human. the detachable urine flow collection device consisting of three areas. a genital ring and opening area, a quick wicking area, and a urine and liquid absorbing area. the purpose of the detachable urine flow collection device is to direct urine and liquids away from the user to a urine and liquid absorbing area of the device. the direction and movement of urine and liquids away from the user, is attained by utilizing specific nonwoven materials in layers, and combinations, to achieve the purpose. the specific properties of the nonwoven materials and the combinations allow this movement and capturing and storage of urine and liquids to take place in the detachable urine flow collection device. the detachable urine flow collection device is designed to fit through an opening in the front portion of an disposable nonwoven garment (diaper) or brief, as well as a traditional underwear garment. the detachable urine flow collection device can be applied or removed from the attachment as needed. the genital ring and opening area of the detachable urine flow collection device assembly of the present invention mates with an opening formed in an absorbent disposable nonwoven or underwear garment. the genital ring and opening area is the top ⅓ of the detachable urine flow collection device. the connection is an intimate contact, and waterproof;
means for the soft moldable nonwoven material of the genital ring and opening is designed to be in full contact and surround the users genitalia. being moldable, the material can be molded and an intimate contact can be made between the users genitals, and the detachable urine flow collection device;
means for the closure ring is a nonwoven material that attaches to the underside of the genital ring and opening area it is a nonwoven with the same ellipsoid opening reflected in its form. the underside of this nonwoven material closure ring is comprised of velcro tape type fasteners, or sticks tape type fasteners, or can also be one of the newer forms of elasticized nonwoven velcro tapes emerging in the absorbent garment (diaper) brief market;
means for the attachment of detachable urine flow collection device to the disposable nonwoven or underwear garment. this is achieved by velcro tape type fasteners, or sticks tape type fasteners on the underside of the nonwoven material closure ring, that adhere to a opposite material on the disposable nonwoven or underwear garment;
means for assistance in the replacement of the detachable urine flow collection device. disposable nonwoven or underwear garment is pulled down to above the knees by the user. the user then removes the detachable urine flow collection device by pulling the tab this breaks the closure between the closure ring and the disposable nonwoven or underwear garment and allows the detachable urine flow collection device to be removed and a new one applied;
means for the separation of urine from the material layers of the genital ring and opening area and the users skin. this is accomplished by this innermost layer of the genital ring and opening area, being a through air bond (tab) nonwoven. the gr layer 1 (through air bond (tab) nonwoven) material allows urine and liquid to pass through it to the next layer of the genital ring and opening area but will not allow it to pass back through the layer and come in contact with the user's skin, structurally connected to said means for the genital ring and opening structurally makes up the top ⅓ of the detachable urine flow collection device and is shaped like an ellipse with the opening, made of a soft moldable nonwoven material. the front of the genital ring is the widest part of the ellipse, and moving backward it ellipses backward to its narrowest part. the closure ring is a nonwoven material that attaches to the top outside edge of the ellipsoid opening, at ninety degrees to the body of the detachable urine flow collection device. the underside of this nonwoven material closure ring is comprised of velcro tape type fasteners, or sticks tape type fasteners, or can also be one of the newer forms of elasticized nonwoven velcro tapes emerging in the absorbent garment (diaper) brief market. the tab a loose flap of nonwoven material will be attached to the font and top of the ellipsoid opening. the genital ring area is made up of the following nonwoven layers, and is described moving from the innermost layer closest to the open chamber of the detachable urine flow collection device moving outward. the innermost first layer is a through air bond (tab) nonwoven that provides separation from any wet area and the skin. the second layer is a hydrophobic nonwoven material. the third layer is a hydrophilic nonwoven material. the fourth and final layer is a polyethylene nonwoven material that is the outside layer of the detachable urine flow collection device, and is entirely hydrophobic or waterproof. the detachable urine flow collection device is designed to fit through an opening in the front portion of an absorbent disposable nonwoven garment (diaper) or brief, as well as a traditional underwear garment (garment). the function of the genital ring and opening is to accommodate the wearers genitals, furthermore the genital ring is the part of the detachable urine flow collection device that accommodates the attachment with the absorbent disposable nonwoven garment through the closure ring. the genital ring and opening of the external collection assembly of the present invention mates with the opening formed in an absorbent disposable nonwoven garment. the connection is an intimate contact, and waterproof. the underside surface of the closure ring, the fastener will make contact with the opposite nonwoven fasteners on the inside surface of the absorbent disposable non-woven garment that lines the opening of the absorbent disposable nonwoven garment. with a user wearing an absorbent disposable nonwoven garment, the widest part of the ellipsoid opening anatomically is toward the front of the user; the opening then ellipses toward the narrowest part, at the back or posterior of the user. the detachable urine flow collection device can be applied or removed from the attachment as needed by pulling on the tab and lifting the detachable urine flow collection device out of the absorbent disposable nonwoven garment. the genital ring or opening top ⅓ of the detachable urine flow collection device then progresses to the quick wicking or middle ⅓ of the detachable urine flow collection device;
means for assisting the movement of urine into and down the genital ring and opening area, and away from the skin of the user. this is accomplished by the elements and composition of a hydrophobic nonwoven material. the hydrophobic (water molecule repellant) nature of this nonwoven layer as well as gravity moves the urine and liquid toward the deeper and outermost layers of the genital ring and opening area, cohesively connected to said means for the separation of urine from the material layers of the genital ring and opening area and the users skin. this is accomplished by this innermost layer of the genital ring and opening area, being a through air bond (tab) nonwoven. the gr layer 1 (through air bond (tab) nonwoven) material allows urine and liquid to pass through it to the next layer of the genital ring and opening area but will not allow it to pass back through the layer and come in contact with the user's skin, and structurally connected to said means for the genital ring and opening structurally makes up the top ⅓ of the detachable urine flow collection device and is shaped like an ellipse with the opening, made of a soft moldable nonwoven material. the front of the genital ring is the widest part of the ellipse, and moving backward it ellipses backward to its narrowest part. the closure ring is a nonwoven material that attaches to the top outside edge of the ellipsoid opening, at ninety degrees to the body of the detachable urine flow collection device. the underside of this nonwoven material closure ring is comprised of velcro tape type fasteners, or sticks tape type fasteners, or can also be one of the newer forms of elasticized nonwoven velcro tapes emerging in the absorbent garment (diaper) brief market. the tab a loose flap of nonwoven material will be attached to the font and top of the ellipsoid opening. the genital ring area is made up of the following nonwoven layers, and is described moving from the innermost layer closest to the open chamber of the detachable urine flow collection device moving outward. the innermost first layer is a through air bond (tab) nonwoven that provides separation from any wet area and the skin. the second layer is a hydrophobic nonwoven material. the third layer is a hydrophilic nonwoven material. the fourth and final layer is a polyethylene nonwoven material that is the outside layer of the detachable urine flow collection device, and is entirely hydrophobic or waterproof. the detachable urine flow collection device is designed to fit through an opening in the front portion of an absorbent disposable nonwoven garment (diaper) or brief, as well as a traditional underwear garment (garment). the function of the genital ring and opening is to accommodate the wearers genitals, furthermore the genital ring is the part of the detachable urine flow collection device that accommodates the attachment with the absorbent disposable nonwoven garment through the closure ring. the genital ring and opening of the external collection assembly of the present invention mates with the opening formed in an absorbent disposable nonwoven garment. the connection is an intimate contact, and waterproof. the underside surface of the closure ring, the fastener will make contact with the opposite nonwoven fasteners on the inside surface of the absorbent disposable non-woven garment that lines the opening of the absorbent disposable nonwoven garment. with a user wearing an absorbent disposable nonwoven garment, the widest part of the ellipsoid opening anatomically is toward the front of the user; the opening then ellipses toward the narrowest part, at the back or posterior of the user. the detachable urine flow collection device can be applied or removed from the attachment as needed by pulling on the tab and lifting the detachable urine flow collection device out of the absorbent disposable nonwoven garment. the genital ring or opening top ⅓ of the detachable urine flow collection device then progresses to the quick wicking or middle ⅓ of the detachable urine flow collection device;
means for moving the urine further and downward outward into the genital ring and opening area. this is accomplished by the elements and composition of a hydrophilic nature of the nonwoven material used in this layer. the hydrophillic (water molecule attractant) nature of this nonwoven layer, as well as gravity moves attracts and traps the urine and liquid toward the deeper and outermost layers of the genital ring and opening area, cohesively connected to said means for assisting the movement of urine into and down the genital ring and opening area, and away from the skin of the user. this is accomplished by the elements and composition of a hydrophobic nonwoven material. the hydrophobic (water molecule repellant) nature of this nonwoven layer as well as gravity moves the urine and liquid toward the deeper and outermost layers of the genital ring and opening area, and structurally connected to said means for the genital ring and opening structurally makes up the top ⅓ of the detachable urine flow collection device and is shaped like an ellipse with the opening, made of a soft moldable nonwoven material. the front of the genital ring is the widest part of the ellipse, and moving backward it ellipses backward to its narrowest part. the closure ring is a nonwoven material that attaches to the top outside edge of the ellipsoid opening, at ninety degrees to the body of the detachable urine flow collection device. the underside of this nonwoven material closure ring is comprised of velcro tape type fasteners, or sticks tape type fasteners, or can also be one of the newer forms of elasticized nonwoven velcro tapes emerging in the absorbent garment (diaper) brief market. the tab a loose flap of nonwoven material will be attached to the font and top of the ellipsoid opening. the genital ring area is made up of the following nonwoven layers, and is described moving from the innermost layer closest to the open chamber of the detachable urine flow collection device moving outward. the innermost first layer is a through air bond (tab) nonwoven that provides separation from any wet area and the skin. the second layer is a hydrophobic nonwoven material. the third layer is a hydrophilic nonwoven material. the fourth and final layer is a polyethylene nonwoven material that is the outside layer of the detachable urine flow collection device, and is entirely hydrophobic or waterproof. the detachable urine flow collection device is designed to fit through an opening in the front portion of an absorbent disposable nonwoven garment (diaper) or brief, as well as a traditional underwear garment (garment). the function of the genital ring and opening is to accommodate the wearers genitals, furthermore the genital ring is the part of the detachable urine flow collection device that accommodates the attachment with the absorbent disposable nonwoven garment through the closure ring. the genital ring and opening of the external collection assembly of the present invention mates with the opening formed in an absorbent disposable nonwoven garment. the connection is an intimate contact, and waterproof. the underside surface of the closure ring, the fastener will make contact with the opposite nonwoven fasteners on the inside surface of the absorbent disposable non-woven garment that lines the opening of the absorbent disposable nonwoven garment. with a user wearing an absorbent disposable nonwoven garment, the widest part of the ellipsoid opening anatomically is toward the front of the user; the opening then ellipses toward the narrowest part, at the back or posterior of the user. the detachable urine flow collection device can be applied or removed from the attachment as needed by pulling on the tab and lifting the detachable urine flow collection device out of the absorbent disposable nonwoven garment. the genital ring or opening top ⅓ of the detachable urine flow collection device then progresses to the quick wicking or middle ⅓ of the detachable urine flow collection device;
means for maintaining a waterproof layer to the fourth and final non woven layer of the genital ring area. the elements and composition of a waterproof nature of the nonwoven material used in this layer prevents urine and liquid from leaking through the genital ring and opening area and wetting the user's clothes, furniture or bed linens, cohesively connected to said means for moving the urine further and downward outward into the genital ring and opening area. this is accomplished by the elements and composition of a hydrophilic nature of the nonwoven material used in this layer. the hydrophillic (water molecule attractant) nature of this nonwoven layer, as well as gravity moves attracts and traps the urine and liquid toward the deeper and outermost layers of the genital ring and opening area, and structurally connected to said means for the genital ring and opening structurally makes up the top ⅓ of the detachable urine flow collection device and is shaped like an ellipse with the opening, made of a soft moldable nonwoven material. the front of the genital ring is the widest part of the ellipse, and moving backward it ellipses backward to its narrowest part. the closure ring is a nonwoven material that attaches to the top outside edge of the ellipsoid opening, at ninety degrees to the body of the detachable urine flow collection device. the underside of this nonwoven material closure ring is comprised of velcro tape type fasteners, or sticks tape type fasteners, or can also be one of the newer forms of elasticized nonwoven velcro tapes emerging in the absorbent garment (diaper) brief market. the tab a loose flap of nonwoven material will be attached to the font and top of the ellipsoid opening. the genital ring area is made up of the following nonwoven layers, and is described moving from the innermost layer closest to the open chamber of the detachable urine flow collection device moving outward. the innermost first layer is a through air bond (tab) nonwoven that provides separation from any wet area and the skin. the second layer is a hydrophobic nonwoven material. the third layer is a hydrophilic nonwoven material. the fourth and final layer is a polyethylene nonwoven material that is the outside layer of the detachable urine flow collection device, and is entirely hydrophobic or waterproof. the detachable urine flow collection device is designed to fit through an opening in the front portion of an absorbent disposable nonwoven garment (diaper) or brief, as well as a traditional underwear garment (garment). the function of the genital ring and opening is to accommodate the wearers genitals, furthermore the genital ring is the part of the detachable urine flow collection device that accommodates the attachment with the absorbent disposable nonwoven garment through the closure ring. the genital ring and opening of the external collection assembly of the present invention mates with the opening formed in an absorbent disposable nonwoven garment. the connection is an intimate contact, and waterproof. the underside surface of the closure ring, the fastener will make contact with the opposite nonwoven fasteners on the inside surface of the absorbent disposable non-woven garment that lines the opening of the absorbent disposable nonwoven garment. with a user wearing an absorbent disposable nonwoven garment, the widest part of the ellipsoid opening anatomically is toward the front of the user; the opening then ellipses toward the narrowest part, at the back or posterior of the user. the detachable urine flow collection device can be applied or removed from the attachment as needed by pulling on the tab and lifting the detachable urine flow collection device out of the absorbent disposable nonwoven garment. the genital ring or opening top ⅓ of the detachable urine flow collection device then progresses to the quick wicking or middle ⅓ of the detachable urine flow collection device;
means for below the genital ring opening area is the quick wicking area middle ⅓ area of the detachable urine flow collection device. the specific purpose of the quick wicking area is to move the urine and liquid as quickly as possible to the urine and liquid collecting area. the quick wicking layer or middle ⅓ area is also ellipsoid in shape and is longer than the genital opening top ⅓ area in length from front to back. the detachable urine flow collection device is also made of layers of nonwoven material that are designed to channel urine and liquid away from the user. these layers act in a specific fashion in order to wick the urine and liquid away from the user to a specific portion of the detachable urine flow collection device. the quick wicking middle ⅓ area nonwoven layers. include the following, and is described moving from the innermost layer closest to the open chamber of the detachable urine flow collection device moving outward. the innermost first layer is a continuation of the first layer of the genital ring and is a through air bond (tab) nonwoven that provides separation from the wet area and the skin. the second layer is a continuation of the hydrophilic nonwoven material from the genital ring. this second layer is pleated and angled back toward the back and bottom of the detachable urine flow collection device to facilitate the movement of urine and liquid toward the bottom ⅓ area; the urine and liquid absorbing area. the third and final layer is a polyethylene nonwoven material that is the outside layer of the entire detachable urine flow collection device, and is entirely hydrophobic or waterproof, sequentially connected to said means for the genital ring and opening structurally makes up the top ⅓ of the detachable urine flow collection device and is shaped like an ellipse with the opening, made of a soft moldable nonwoven material. the front of the genital ring is the widest part of the ellipse, and moving backward it ellipses backward to its narrowest part. the closure ring is a nonwoven material that attaches to the top outside edge of the ellipsoid opening, at ninety degrees to the body of the detachable urine flow collection device. the underside of this nonwoven material closure ring is comprised of velcro tape type fasteners, or sticks tape type fasteners, or can also be one of the newer forms of elasticized nonwoven velcro tapes emerging in the absorbent garment (diaper) brief market. the tab a loose flap of nonwoven material will be attached to the font and top of the ellipsoid opening. the genital ring area is made up of the following nonwoven layers, and is described moving from the innermost layer closest to the open chamber of the detachable urine flow collection device moving outward. the innermost first layer is a through air bond (tab) nonwoven that provides separation from any wet area and the skin. the second layer is a hydrophobic nonwoven material. the third layer is a hydrophilic nonwoven material. the fourth and final layer is a polyethylene nonwoven material that is the outside layer of the detachable urine flow collection device, and is entirely hydrophobic or waterproof. the detachable urine flow collection device is designed to fit through an opening in the front portion of an absorbent disposable nonwoven garment (diaper) or brief, as well as a traditional underwear garment (garment). the function of the genital ring and opening is to accommodate the wearers genitals, furthermore the genital ring is the part of the detachable urine flow collection device that accommodates the attachment with the absorbent disposable nonwoven garment through the closure ring. the genital ring and opening of the external collection assembly of the present invention mates with the opening formed in an absorbent disposable nonwoven garment. the connection is an intimate contact, and waterproof. the underside surface of the closure ring, the fastener will make contact with the opposite nonwoven fasteners on the inside surface of the absorbent disposable non-woven garment that lines the opening of the absorbent disposable nonwoven garment. with a user wearing an absorbent disposable nonwoven garment, the widest part of the ellipsoid opening anatomically is toward the front of the user; the opening then ellipses toward the narrowest part, at the back or posterior of the user. the detachable urine flow collection device can be applied or removed from the attachment as needed by pulling on the tab and lifting the detachable urine flow collection device out of the absorbent disposable nonwoven garment. the genital ring or opening top ⅓ of the detachable urine flow collection device then progresses to the quick wicking or middle ⅓ of the detachable urine flow collection device, and structurally connected to said means for a detachable urine flow collection device that can be attached to an absorbent garment (diaper) brief or traditional underwear garment (garment) worn by a human. the detachable urine flow collection device consisting of three areas. a genital ring and opening area, a quick wicking area, and a urine and liquid absorbing area. the purpose of the detachable urine flow collection device is to direct urine and liquids away from the user to a urine and liquid absorbing area of the device. the direction and movement of urine and liquids away from the user, is attained by utilizing specific nonwoven materials in layers, and combinations, to achieve the purpose. the specific properties of the nonwoven materials and the combinations allow this movement and capturing and storage of urine and liquids to take place in the detachable urine flow collection device. the detachable urine flow collection device is designed to fit through an opening in the front portion of an disposable nonwoven garment (diaper) or brief, as well as a traditional underwear garment. the detachable urine flow collection device can be applied or removed from the attachment as needed. the genital ring and opening area of the detachable urine flow collection device assembly of the present invention mates with an opening formed in an absorbent disposable nonwoven or underwear garment. the genital ring and opening area is the top ⅓ of the detachable urine flow collection device. the connection is an intimate contact, and waterproof;
means for the separation of urine from the material layers of the quick wicking area and the users skin. this is accomplished by this innermost layer of the quick wicking area, being a continuation of the above innermost layer of the genital ring and is a through air bond (tab) nonwoven. the qw layer 1 (through air bond (tab) nonwoven) material allows urine and liquid to pass through it to the next layer of the quick wicking area but will not allow it to pass back through the layer and come in contact with the user's skin, structurally connected to said means for below the genital ring opening area is the quick wicking area middle ⅓ area of the detachable urine flow collection device. the specific purpose of the quick wicking area is to move the urine and liquid as quickly as possible to the urine and liquid collecting area. the quick wicking layer or middle ⅓ area is also ellipsoid in shape and is longer than the genital opening top ⅓ area in length from front to back. the detachable urine flow collection device is also made of layers of nonwoven material that are designed to channel urine and liquid away from the user. these layers act in a specific fashion in order to wick the urine and liquid away from the user to a specific portion of the detachable urine flow collection device. the quick wicking middle ⅓ area nonwoven layers. include the following, and is described moving from the innermost layer closest to the open chamber of the detachable urine flow collection device moving outward. the innermost first layer is a continuation of the first layer of the genital ring and is a through air bond (tab) nonwoven that provides separation from the wet area and the skin. the second layer is a continuation of the hydrophilic nonwoven material from the genital ring. this second layer is pleated and angled back toward the back and bottom of the detachable urine flow collection device to facilitate the movement of urine and liquid toward the bottom ⅓ area; the urine and liquid absorbing area. the third and final layer is a polyethylene nonwoven material that is the outside layer of the entire detachable urine flow collection device, and is entirely hydrophobic or waterproof, and sequentially connected to said means for the separation of urine from the material layers of the genital ring and opening area and the users skin. this is accomplished by this innermost layer of the genital ring and opening area, being a through air bond (tab) nonwoven. the gr layer 1 (through air bond (tab) nonwoven) material allows urine and liquid to pass through it to the next layer of the genital ring and opening area but will not allow it to pass back through the layer and come in contact with the user's skin;
means for moving the urine further and downward outward into the quick wicking area. this is accomplished by this layer being a continuation of the above layer of the genital ring and opening area and the elements and composition of a hydrophilic nature of the nonwoven material used in this layer. the hydrophillic (water molecule attractant) nature of this nonwoven layer, as well as gravity moves attracts and traps the urine and liquid toward the deeper and outermost layers of the genital ring and opening area, cohesively connected to said means for the separation of urine from the material layers of the quick wicking area and the users skin. this is accomplished by this innermost layer of the quick wicking area, being a continuation of the above innermost layer of the genital ring and is a through air bond (tab) nonwoven. the qw layer 1 (through air bond (tab) nonwoven) material allows urine and liquid to pass through it to the next layer of the quick wicking area but will not allow it to pass back through the layer and come in contact with the user's skin, structurally connected to said means for below the genital ring opening area is the quick wicking area middle ⅓ area of the detachable urine flow collection device. the specific purpose of the quick wicking area is to move the urine and liquid as quickly as possible to the urine and liquid collecting area. the quick wicking layer or middle ⅓ area is also ellipsoid in shape and is longer than the genital opening top ⅓ area in length from front to back. the detachable urine flow collection device is also made of layers of nonwoven material that are designed to channel urine and liquid away from the user. these layers act in a specific fashion in order to wick the urine and liquid away from the user to a specific portion of the detachable urine flow collection device. the quick wicking middle ⅓ area nonwoven layers. include the following, and is described moving from the innermost layer closest to the open chamber of the detachable urine flow collection device moving outward. the innermost first layer is a continuation of the first layer of the genital ring and is a through air bond (tab) nonwoven that provides separation from the wet area and the skin. the second layer is a continuation of the hydrophilic nonwoven material from the genital ring. this second layer is pleated and angled back toward the back and bottom of the detachable urine flow collection device to facilitate the movement of urine and liquid toward the bottom ⅓ area; the urine and liquid absorbing area. the third and final layer is a polyethylene nonwoven material that is the outside layer of the entire detachable urine flow collection device, and is entirely hydrophobic or waterproof, and sequentially connected to said means for moving the urine further and downward outward into the genital ring and opening area. this is accomplished by the elements and composition of a hydrophilic nature of the nonwoven material used in this layer. the hydrophillic (water molecule attractant) nature of this nonwoven layer, as well as gravity moves attracts and traps the urine and liquid toward the deeper and outermost layers of the genital ring and opening area;
means for maintaining a waterproof layer to the fourth and final non woven layer of the wicking area. this layer is a continuation of the above layer of the genital ring and opening area. the elements and composition of a waterproof nature of the nonwoven material used in this layer prevents urine and liquid from leaking through the quick wicking area and wetting the user's clothes, furniture or bed linens, cohesively connected to said means for moving the urine further and downward outward into the quick wicking area. this is accomplished by this layer being a continuation of the above layer of the genital ring and opening area and the elements and composition of a hydrophilic nature of the nonwoven material used in this layer. the hydrophillic (water molecule attractant) nature of this nonwoven layer, as well as gravity moves attracts and traps the urine and liquid toward the deeper and outermost layers of the genital ring and opening area, structurally connected to said means for below the genital ring opening area is the quick wicking area middle ⅓ area of the detachable urine flow collection device. the specific purpose of the quick wicking area is to move the urine and liquid as quickly as possible to the urine and liquid collecting area. the quick wicking layer or middle ⅓ area is also ellipsoid in shape and is longer than the genital opening top ⅓ area in length from front to back. the detachable urine flow collection device is also made of layers of nonwoven material that are designed to channel urine and liquid away from the user. these layers act in a specific fashion in order to wick the urine and liquid away from the user to a specific portion of the detachable urine flow collection device. the quick wicking middle ⅓ area nonwoven layers. include the following, and is described moving from the innermost layer closest to the open chamber of the detachable urine flow collection device moving outward. the innermost first layer is a continuation of the first layer of the genital ring and is a through air bond (tab) nonwoven that provides separation from the wet area and the skin. the second layer is a continuation of the hydrophilic nonwoven material from the genital ring. this second layer is pleated and angled back toward the back and bottom of the detachable urine flow collection device to facilitate the movement of urine and liquid toward the bottom ⅓ area; the urine and liquid absorbing area. the third and final layer is a polyethylene nonwoven material that is the outside layer of the entire detachable urine flow collection device, and is entirely hydrophobic or waterproof, and sequentially connected to said means for maintaining a waterproof layer to the fourth and final non woven layer of the genital ring area. the elements and composition of a waterproof nature of the nonwoven material used in this layer prevents urine and liquid from leaking through the genital ring and opening area and wetting the user's clothes, furniture or bed linens;
means for the urine and liquid absorbing area, bottom ⅓ area is also ellipsoid in shape and is approximately the same size as the quick wicking layer or middle ⅓ area in length from front to back. the urine and liquid absorbing area, is made up of the following layers, and is described moving from the innermost layer closest to the open chamber of the detachable urine flow collection device moving outward. the innermost first layer is a continuation of the first layer of the quick wicking area and is a through air bond (tab) nonwoven that provides separation from the wet area and the skin. the second layer is a continuation of the hydrophillic nonwoven material from the quick wicking area. the third layer is the core made up of wood pulp or cellulose which really is the core of the detachable urine flow collection device to absorb the urine and liquids. this provides the absorbing capacity of the detachable urine flow collection device. super absorbent polymer-sodium polyacrylate, (sap) is interwoven throughout the cellulose to allow the core to absorb urine and more liquid and maintain control of this liquid when the core is under pressure. the liberal use of sap in this layer, allows less wood pulp or cellulose to be used thus creating a more streamlined garment. the fourth layer and final layer is a polyethylene non-woven material that is the outside layer of the entire detachable urine flow collection device, and is entirely hydrophobic or waterproof, sequentially connected to said means for below the genital ring opening area is the quick wicking area middle ⅓ area of the detachable urine flow collection device. the specific purpose of the quick wicking area is to move the urine and liquid as quickly as possible to the urine and liquid collecting area. the quick wicking layer or middle ⅓ area is also ellipsoid in shape and is longer than the genital opening top ⅓ area in length from front to back. the detachable urine flow collection device is also made of layers of nonwoven material that are designed to channel urine and liquid away from the user. these layers act in a specific fashion in order to wick the urine and liquid away from the user to a specific portion of the detachable urine flow collection device. the quick wicking middle ⅓ area nonwoven layers. include the following, and is described moving from the innermost layer closest to the open chamber of the detachable urine flow collection device moving outward. the innermost first layer is a continuation of the first layer of the genital ring and is a through air bond (tab) nonwoven that provides separation from the wet area and the skin. the second layer is a continuation of the hydrophilic nonwoven material from the genital ring. this second layer is pleated and angled back toward the back and bottom of the detachable urine flow collection device to facilitate the movement of urine and liquid toward the bottom ⅓ area; the urine and liquid absorbing area. the third and final layer is a polyethylene nonwoven material that is the outside layer of the entire detachable urine flow collection device, and is entirely hydrophobic or waterproof, and structurally connected to said means for a detachable urine flow collection device that can be attached to an absorbent garment (diaper) brief or traditional underwear garment (garment) worn by a human. the detachable urine flow collection device consisting of three areas. a genital ring and opening area, a quick wicking area, and a urine and liquid absorbing area. the purpose of the detachable urine flow collection device is to direct urine and liquids away from the user to a urine and liquid absorbing area of the device. the direction and movement of urine and liquids away from the user, is attained by utilizing specific nonwoven materials in layers, and combinations, to achieve the purpose. the specific properties of the nonwoven materials and the combinations allow this movement and capturing and storage of urine and liquids to take place in the detachable urine flow collection device. the detachable urine flow collection device is designed to fit through an opening in the front portion of an disposable nonwoven garment (diaper) or brief, as well as a traditional underwear garment. the detachable urine flow collection device can be applied or removed from the attachment as needed. the genital ring and opening area of the detachable urine flow collection device assembly of the present invention mates with an opening formed in an absorbent disposable nonwoven or underwear garment. the genital ring and opening area is the top ⅓ of the detachable urine flow collection device. the connection is an intimate contact, and waterproof;
means for the separation of urine from the material layers of the urine and liquid absorbing area and the users skin. this is accomplished by this innermost layer of the urine and liquid absorbing area, being a continuation of the above innermost layer of the quick wicking area and is a through air bond (tab) nonwoven. the qw layer 1 (through air bond (tab) nonwoven) material allows urine and liquid to pass through it to the next layer of the ui layer 1 (through air bond (tab) nonwoven), but will not allow it to pass back through the layer and come in contact with the user's skin, structurally connected to said means for the urine and liquid absorbing area, bottom ⅓ area is also ellipsoid in shape and is approximately the same size as the quick wicking layer or middle ⅓ area in length from front to back. the urine and liquid absorbing area, is made up of the following layers, and is described moving from the innermost layer closest to the open chamber of the detachable urine flow collection device moving outward. the innermost first layer is a continuation of the first layer of the quick wicking area and is a through air bond (tab) nonwoven that provides separation from the wet area and the skin. the second layer is a continuation of the hydrophillic nonwoven material from the quick wicking area. the third layer is the core made up of wood pulp or cellulose which really is the core of the detachable urine flow collection device to absorb the urine and liquids. this provides the absorbing capacity of the detachable urine flow collection device. super absorbent polymer-sodium polyacrylate, (sap) is interwoven throughout the cellulose to allow the core to absorb urine and more liquid and maintain control of this liquid when the core is under pressure. the liberal use of sap in this layer, allows less wood pulp or cellulose to be used thus creating a more streamlined garment. the fourth layer and final layer is a polyethylene non-woven material that is the outside layer of the entire detachable urine flow collection device, and is entirely hydrophobic or waterproof, and sequentially connected to said means for the separation of urine from the material layers of the quick wicking area and the users skin. this is accomplished by this innermost layer of the quick wicking area, being a continuation of the above innermost layer of the genital ring and is a through air bond (tab) nonwoven. the qw layer 1 (through air bond (tab) nonwoven) material allows urine and liquid to pass through it to the next layer of the quick wicking area but will not allow it to pass back through the layer and come in contact with the user's skin;
means for moving the urine further and downward outward into the urine and liquid absorbing area. this is accomplished by this layer being a continuation of the above layer of the quick wicking area and the elements and composition of a hydrophilic nature of the nonwoven material used in this layer. the hydrophillic (water molecule attractant) nature of this nonwoven layer, as well as gravity moves, attracts and traps the urine and liquid toward the deeper and outermost layers of the urine and liquid absorbing area, cohesively connected to said means for the separation of urine from the material layers of the urine and liquid absorbing area and the users skin. this is accomplished by this innermost layer of the urine and liquid absorbing area, being a continuation of the above innermost layer of the quick wicking area and is a through air bond (tab) nonwoven. the qw layer 1 (through air bond (tab) nonwoven) material allows urine and liquid to pass through it to the next layer of the ui layer 1 (through air bond (tab) nonwoven), but will not allow it to pass back through the layer and come in contact with the user's skin, structurally connected to said means for the urine and liquid absorbing area, bottom ⅓ area is also ellipsoid in shape and is approximately the same size as the quick wicking layer or middle ⅓ area in length from front to back. the urine and liquid absorbing area, is made up of the following layers, and is described moving from the innermost layer closest to the open chamber of the detachable urine flow collection device moving outward. the innermost first layer is a continuation of the first layer of the quick wicking area and is a through air bond (tab) nonwoven that provides separation from the wet area and the skin. the second layer is a continuation of the hydrophillic nonwoven material from the quick wicking area. the third layer is the core made up of wood pulp or cellulose which really is the core of the detachable urine flow collection device to absorb the urine and liquids. this provides the absorbing capacity of the detachable urine flow collection device. super absorbent polymer-sodium polyacrylate, (sap) is interwoven throughout the cellulose to allow the core to absorb urine and more liquid and maintain control of this liquid when the core is under pressure. the liberal use of sap in this layer, allows less wood pulp or cellulose to be used thus creating a more streamlined garment. the fourth layer and final layer is a polyethylene non-woven material that is the outside layer of the entire detachable urine flow collection device, and is entirely hydrophobic or waterproof, and sequentially connected to said means for moving the urine further and downward outward into the quick wicking area. this is accomplished by this layer being a continuation of the above layer of the genital ring and opening area and the elements and composition of a hydrophilic nature of the nonwoven material used in this layer. the hydrophillic (water molecule attractant) nature of this nonwoven layer, as well as gravity moves attracts and traps the urine and liquid toward the deeper and outermost layers of the genital ring and opening area;
means for the adsorption and retention of liquid and urine in the urine and liquid absorbing area. the third layer of the urine and liquid absorbing area is the core made up of wood pulp or cellulose which really is the core of the detachable urine flow collection device to absorb the urine and liquids. this provides the absorbing capacity of the detachable urine flow collection device. super absorbent polymer-sodium polyacrylate, (sap) is interwoven throughout the cellulose to allow the core to absorb urine and more liquid and maintain control of this liquid when the core is under pressure. the liberal use of sap in this layer, allows less wood pulp or cellulose to be used thus creating a more streamlined garment, cohesively connected to said means for moving the urine further and downward outward into the urine and liquid absorbing area. this is accomplished by this layer being a continuation of the above layer of the quick wicking area and the elements and composition of a hydrophilic nature of the nonwoven material used in this layer. the hydrophillic (water molecule attractant) nature of this nonwoven layer, as well as gravity moves, attracts and traps the urine and liquid toward the deeper and outermost layers of the urine and liquid absorbing area, and structurally connected to said means for the urine and liquid absorbing area, bottom ⅓ area is also ellipsoid in shape and is approximately the same size as the quick wicking layer or middle ⅓ area in length from front to back. the urine and liquid absorbing area, is made up of the following layers, and is described moving from the innermost layer closest to the open chamber of the detachable urine flow collection device moving outward. the innermost first layer is a continuation of the first layer of the quick wicking area and is a through air bond (tab) nonwoven that provides separation from the wet area and the skin. the second layer is a continuation of the hydrophillic nonwoven material from the quick wicking area. the third layer is the core made up of wood pulp or cellulose which really is the core of the detachable urine flow collection device to absorb the urine and liquids. this provides the absorbing capacity of the detachable urine flow collection device. super absorbent polymer-sodium polyacrylate, (sap) is interwoven throughout the cellulose to allow the core to absorb urine and more liquid and maintain control of this liquid when the core is under pressure. the liberal use of sap in this layer, allows less wood pulp or cellulose to be used thus creating a more streamlined garment. the fourth layer and final layer is a polyethylene non-woven material that is the outside layer of the entire detachable urine flow collection device, and is entirely hydrophobic or waterproof; and
means for maintaining a waterproof layer to the fourth and final non woven layer of the urine and liquid absorbing area. this layer is a continuation of the above layer of the quick wicking area. the elements and composition of a waterproof nature of the nonwoven material used in this layer prevents urine and liquid from leaking through the urine and liquid absorbing area and wetting the user's clothes, furniture or bed linens, cohesively connected to said means for the adsorption and retention of liquid and urine in the urine and liquid absorbing area. the third layer of the urine and liquid absorbing area is the core made up of wood pulp or cellulose which really is the core of the detachable urine flow collection device to absorb the urine and liquids. this provides the absorbing capacity of the detachable urine flow collection device. super absorbent polymer-sodium polyacrylate, (sap) is interwoven throughout the cellulose to allow the core to absorb urine and more liquid and maintain control of this liquid when the core is under pressure. the liberal use of sap in this layer, allows less wood pulp or cellulose to be used thus creating a more streamlined garment, structurally connected to said means for the urine and liquid absorbing area, bottom ⅓ area is also ellipsoid in shape and is approximately the same size as the quick wicking layer or middle ⅓ area in length from front to back. the urine and liquid absorbing area, is made up of the following layers, and is described moving from the innermost layer closest to the open chamber of the detachable urine flow collection device moving outward. the innermost first layer is a continuation of the first layer of the quick wicking area and is a through air bond (tab) nonwoven that provides separation from the wet area and the skin. the second layer is a continuation of the hydrophillic nonwoven material from the quick wicking area. the third layer is the core made up of wood pulp or cellulose which really is the core of the detachable urine flow collection device to absorb the urine and liquids. this provides the absorbing capacity of the detachable urine flow collection device. super absorbent polymer-sodium polyacrylate, (sap) is interwoven throughout the cellulose to allow the core to absorb urine and more liquid and maintain control of this liquid when the core is under pressure. the liberal use of sap in this layer, allows less wood pulp or cellulose to be used thus creating a more streamlined garment. the fourth layer and final layer is a polyethylene non-woven material that is the outside layer of the entire detachable urine flow collection device, and is entirely hydrophobic or waterproof, and sequentially connected to said means for maintaining a waterproof layer to the fourth and final non woven layer of the wicking area. this layer is a continuation of the above layer of the genital ring and opening area. the elements and composition of a waterproof nature of the nonwoven material used in this layer prevents urine and liquid from leaking through the quick wicking area and wetting the user's clothes, furniture or bed linens.

2. The human wearable and detachable incontinence device in accordance with claim 1, wherein said means for a detachable urine flow collection device that can be attached to an absorbent garment (diaper) brief or traditional underwear garment (garment) worn by a human. the detachable urine flow collection device consisting of three areas. a genital ring and opening area, a quick wicking area, and a urine and liquid absorbing area. the purpose of the detachable urine flow collection device is to direct urine and liquids away from the user to a urine and liquid absorbing area of the device. the direction and movement of urine and liquids away from the user, is attained by utilizing specific nonwoven materials in layers, and combinations, to achieve the purpose. the specific properties of the nonwoven materials and the combinations allow this movement and capturing and storage of urine and liquids to take place in the detachable urine flow collection device. the detachable urine flow collection device is designed to fit through an opening in the front portion of an disposable nonwoven garment (diaper) or brief, as well as a traditional underwear garment. the detachable urine flow collection device can be applied or removed from the attachment as needed. the genital ring and opening area of the detachable urine flow collection device assembly of the present invention mates with an opening formed in an absorbent disposable nonwoven or underwear garment. the genital ring and opening area is the top ⅓ of the detachable urine flow collection device. the connection is an intimate contact, and waterproof comprises an ellipsoid in shape, front is the widest part of the ellipse shape, back part is the narrowest part of the ellipse shape, ellipsoid shape maintained in high top to bottom, consists 3 areas, non-woven materials perform different roles in the different areas detachable urine flow collection device.

3. The human wearable and detachable incontinence device in accordance with claim 1, wherein said means for the application of the detachable urine flow collection device. the detachable urine flow collection device fits through the garment opening in the front anterior portion of the disposable nonwoven or underwear garment. the detachable urine flow collection device attaches to the disposable nonwoven or underwear garment by velcro tape or stick type fasteners on the underside surface of the closure ring of the urine and liquid absorbing area comprises a disposable, slit opening in the front anterior panel disposable nonwoven or underwear garment.

4. The human wearable and detachable incontinence device in accordance with claim 1, wherein said means for the application of the detachable urine flow collection device to the disposable nonwoven or underwear garment. the detachable urine flow collection device fits through the garment opening in the front anterior portion of the disposable nonwoven or underwear garment. the detachable urine flow collection device attaches to the disposable nonwoven or underwear garment by velcro tape or stick type fasteners on the underside surface of the closure ring of the urine and liquid absorbing area. these velcro tape or stick type fasteners on the underside of the closure ring attached directly to the opposite material on the inside surface of the disposable nonwoven or underwear garment opening comprises a slit opening in the ant front of the disposable nonwoven or underwear garment, top inside surface opposite material velcro tape or stick type fasteners garment opening.

5. The human wearable and detachable incontinence device in accordance with claim 1, wherein said means for the genital ring and opening structurally makes up the top ⅓ of the detachable urine flow collection device and is shaped like an ellipse with the opening, made of a soft moldable nonwoven material. the front of the genital ring is the widest part of the ellipse, and moving backward it ellipses backward to its narrowest part. the closure ring is a nonwoven material that attaches to the top outside edge of the ellipsoid opening, at ninety degrees to the body of the detachable urine flow collection device. the underside of this nonwoven material closure ring is comprised of velcro tape type fasteners, or sticks tape type fasteners, or can also be one of the newer forms of elasticized nonwoven velcro tapes emerging in the absorbent garment (diaper) brief market. the tab a loose flap of nonwoven material will be attached to the font and top of the ellipsoid opening. the genital ring area is made up of the following nonwoven layers, and is described moving from the innermost layer closest to the open chamber of the detachable urine flow collection device moving outward. the innermost first layer is a through air bond (tab) nonwoven that provides separation from any wet area and the skin. the second layer is a hydrophobic nonwoven material. the third layer is a hydrophilic nonwoven material. the fourth and final layer is a polyethylene nonwoven material that is the outside layer of the detachable urine flow collection device, and is entirely hydrophobic or waterproof. the detachable urine flow collection device is designed to fit through an opening in the front portion of an absorbent disposable nonwoven garment (diaper) or brief, as well as a traditional underwear garment (garment). the function of the genital ring and opening is to accommodate the wearers genitals, furthermore the genital ring is the part of the detachable urine flow collection device that accommodates the attachment with the absorbent disposable nonwoven garment through the closure ring. the genital ring and opening of the external collection assembly of the present invention mates with the opening formed in an absorbent disposable nonwoven garment. the connection is an intimate contact, and waterproof. the underside surface of the closure ring, the fastener will make contact with the opposite nonwoven fasteners on the inside surface of the absorbent disposable non-woven garment that lines the opening of the absorbent disposable nonwoven garment. with a user wearing an absorbent disposable nonwoven garment, the widest part of the ellipsoid opening anatomically is toward the front of the user; the opening then ellipses toward the narrowest part, at the back or posterior of the user. the detachable urine flow collection device can be applied or removed from the attachment as needed by pulling on the tab and lifting the detachable urine flow collection device out of the absorbent disposable nonwoven garment. the genital ring or opening top ⅓ of the detachable urine flow collection device then progresses to the quick wicking or middle ⅓ of the detachable urine flow collection device comprises a top ⅓ of the detachable urine flow collection device, soft moldable nonwoven material, forms a seal around the genitals, made of nonwoven material in layers, new attribute 5 genital ring and opening area.

6. The human wearable and detachable incontinence device in accordance with claim 1, wherein said means for the soft moldable nonwoven material of the genital ring and opening is designed to be in full contact and surround the users genitalia. being moldable, the material can be molded and an intimate contact can be made between the users genitals, and the detachable urine flow collection device comprises a soft moldable, encloses and surrounds the genitals soft moldable nonwoven material.

7. The human wearable and detachable incontinence device in accordance with claim 1, wherein said means for the closure ring is a nonwoven material that attaches to the underside of the genital ring and opening area it is a nonwoven with the same ellipsoid opening reflected in its form. the underside of this nonwoven material closure ring is comprised of velcro tape type fasteners, or sticks tape type fasteners, or can also be one of the newer forms of elasticized nonwoven velcro tapes emerging in the absorbent garment (diaper) brief market comprises a non woven material, underside surface velcro tape or stick type fasteners closure ring.

8. The human wearable and detachable incontinence device in accordance with claim 1, wherein said means for the attachment of detachable urine flow collection device to the disposable nonwoven or underwear garment. this is achieved by velcro tape type fasteners, or sticks tape type fasteners on the underside of the nonwoven material closure ring, that adhere to a opposite material on the disposable nonwoven or underwear garment comprises a velcro tape or stick type fasteners, follows the shape of the genital ring and opening area velcro tape or stick type fasteners.

9. The human wearable and detachable incontinence device in accordance with claim 1, wherein said means for assistance in the replacement of the detachable urine flow collection device. disposable nonwoven or underwear garment is pulled down to above the knees by the user. the user then removes the detachable urine flow collection device by pulling the tab this breaks the closure between the closure ring and the disposable nonwoven or underwear garment and allows the detachable urine flow collection device to be removed and a new one applied comprises a non woven material tab.

10. The human wearable and detachable incontinence device in accordance with claim 1, wherein said means for the separation of urine from the material layers of the genital ring and opening area and the users skin. this is accomplished by this innermost layer of the genital ring and opening area, being a through air bond (tab) nonwoven. the gr layer 1 (through air bond (tab) nonwoven) material allows urine and liquid to pass through it to the next layer of the genital ring and opening area but will not allow it to pass back through the layer and come in contact with the user's skin comprises a non woven material, through air bonded, aids in single direction of urine and liquid flo gr layer 1 (through air bond (tab) nonwoven).

11. The human wearable and detachable incontinence device in accordance with claim 1, wherein said means for assisting the movement of urine into and down the genital ring and opening area, and away from the skin of the user. this is accomplished by the elements and composition of a hydrophobic nonwoven material. the hydrophobic (water molecule repellant) nature of this nonwoven layer as well as gravity moves the urine and liquid toward the deeper and outermost layers of the genital ring and opening area comprises a non woven, hydrophobic (water molecule repellant), aids directional flow of urine and liquid gr layer 2 (hydrophobic nonwoven material).

12. The human wearable and detachable incontinence device in accordance with claim 1, wherein said means for moving the urine further and downward outward into the genital ring and opening area. this is accomplished by the elements and composition of a hydrophilic nature of the nonwoven material used in this layer. the hydrophillic (water molecule attractant) nature of this nonwoven layer, as well as gravity moves attracts and traps the urine and liquid toward the deeper and outermost layers of the genital ring and opening area comprises a non woven, hydrophilic (water molecule repellant), aids in the directional flow of urine and liquid gr layer 3 (hydrophilic nonwoven material).

13. The human wearable and detachable incontinence device in accordance with claim 1, wherein said means for maintaining a waterproof layer to the fourth and final non woven layer of the genital ring area. the elements and composition of a waterproof nature of the nonwoven material used in this layer prevents urine and liquid from leaking through the genital ring and opening area and wetting the user's clothes, furniture or bed linens comprises a non woven, waterproof gr layer 4 (polyethylene nonwoven material).

14. The human wearable and detachable incontinence device in accordance with claim 1, wherein said means for below the genital ring opening area is the quick wicking area middle ⅓ area of the detachable urine flow collection device. the specific purpose of the quick wicking area is to move the urine and liquid as quickly as possible to the urine and liquid collecting area. the quick wicking layer or middle ⅓ area is also ellipsoid in shape and is longer than the genital opening top ⅓ area in length from front to back. the detachable urine flow collection device is also made of layers of nonwoven material that are designed to channel urine and liquid away from the user. these layers act in a specific fashion in order to wick the urine and liquid away from the user to a specific portion of the detachable urine flow collection device. the quick wicking middle ⅓ area nonwoven layers. include the following, and is described moving from the innermost layer closest to the open chamber of the detachable urine flow collection device moving outward. the innermost first layer is a continuation of the first layer of the genital ring and is a through air bond (tab) nonwoven that provides separation from the wet area and the skin. the second layer is a continuation of the hydrophilic nonwoven material from the genital ring. this second layer is pleated and angled back toward the back and bottom of the detachable urine flow collection device to facilitate the movement of urine and liquid toward the bottom ⅓ area; the urine and liquid absorbing area. the third and final layer is a polyethylene nonwoven material that is the outside layer of the entire detachable urine flow collection device, and is entirely hydrophobic or waterproof comprises a middle ⅓ of detachable urine flow collection device, ellipsoid opening, non woven layers, directs urine and liquid quick wicking area.

15. The human wearable and detachable incontinence device in accordance with claim 1, wherein said means for the separation of urine from the material layers of the quick wicking area and the users skin. this is accomplished by this innermost layer of the quick wicking area, being a continuation of the above innermost layer of the genital ring and is a through air bond (tab) nonwoven. the qw layer 1 (through air bond (tab) nonwoven) material allows urine and liquid to pass through it to the next layer of the quick wicking area but will not allow it to pass back through the layer and come in contact with the user's skin comprises a non woven, through air bonded, continuation of gr layer 1 (through air bond (tab) nonwoven) above, aids in single direction of urine and liquid flo qw layer 1 (through air bond (tab) nonwoven).

16. The human wearable and detachable incontinence device in accordance with claim 1, wherein said means for moving the urine further and downward outward into the quick wicking area. this is accomplished by this layer being a continuation of the above layer of the genital ring and opening area and the elements and composition of a hydrophilic nature of the nonwoven material used in this layer. the hydrophillic (water molecule attractant) nature of this nonwoven layer, as well as gravity moves attracts and traps the urine and liquid toward the deeper and outermost layers of the genital ring and opening area comprises a non woven, continuation of gr layer 3 (hydrophilic nonwoven material), aids in the directional flow of urine and liquid qw layer 2 (hydrophilic nonwoven material).

17. The human wearable and detachable incontinence device in accordance with claim 1, wherein said means for maintaining a waterproof layer to the fourth and final non woven layer of the wicking area. this layer is a continuation of the above layer of the genital ring and opening area. the elements and composition of a waterproof nature of the nonwoven material used in this layer prevents urine and liquid from leaking through the quick wicking area and wetting the user's clothes, furniture or bed linens comprises a non woven, waterproof, continuation of gr layer 4 (polyethylene nonwoven material) qw layer 3 (polyethylene nonwoven material).

18. The human wearable and detachable incontinence device in accordance with claim 1, wherein said means for the urine and liquid absorbing area, bottom ⅓ area is also ellipsoid in shape and is approximately the same size as the quick wicking layer or middle ⅓ area in length from front to back. the urine and liquid absorbing area, is made up of the following layers, and is described moving from the innermost layer closest to the open chamber of the detachable urine flow collection device moving outward. the innermost first layer is a continuation of the first layer of the quick wicking area and is a through air bond (tab) nonwoven that provides separation from the wet area and the skin. the second layer is a continuation of the hydrophillic nonwoven material from the quick wicking area. the third layer is the core made up of wood pulp or cellulose which really is the core of the detachable urine flow collection device to absorb the urine and liquids. this provides the absorbing capacity of the detachable urine flow collection device. super absorbent polymer-sodium polyacrylate, (sap) is interwoven throughout the cellulose to allow the core to absorb urine and more liquid and maintain control of this liquid when the core is under pressure. the liberal use of sap in this layer, allows less wood pulp or cellulose to be used thus creating a more streamlined garment. the fourth layer and final layer is a polyethylene non-woven material that is the outside layer of the entire detachable urine flow collection device, and is entirely hydrophobic or waterproof comprises a bottom ⅓ of detachable urine flow collection device, ellipsoid opening, non woven layers, directs urine and liquid urine and liquid absorbing area.

19. The human wearable and detachable incontinence device in accordance with claim 1, wherein said means for the separation of urine from the material layers of the urine and liquid absorbing area and the users skin. this is accomplished by this innermost layer of the urine and liquid absorbing area, being a continuation of the above innermost layer of the quick wicking area and is a through air bond (tab) nonwoven. the qw layer 1 (through air bond (tab) nonwoven) material allows urine and liquid to pass through it to the next layer of the ui layer 1 (through air bond (tab) nonwoven), but will not allow it to pass back through the layer and come in contact with the user's skin comprises a non woven, through air bonded, continuation of qw layer 1 (through air bond (tab) nonwoven) above, aids in single direction of urine and liquid flo ul layer 1 (through air bond (tab) nonwoven).

20. The human wearable and detachable incontinence device in accordance with claim 1, wherein said means for moving the urine further and downward outward into the urine and liquid absorbing area. this is accomplished by this layer being a continuation of the above layer of the quick wicking area and the elements and composition of a hydrophilic nature of the nonwoven material used in this layer. the hydrophillic (water molecule attractant) nature of this nonwoven layer, as well as gravity moves, attracts and traps the urine and liquid toward the deeper and outermost layers of the urine and liquid absorbing area comprises a nonwoven, continuation of qw layer 2 (hydrophilic nonwoven material), aids in the directional flow of urine and liquid ul layer 2 (hydrophilic nonwoven material).

21. The human wearable and detachable incontinence device in accordance with claim 1, wherein said means for the adsorption and retention of liquid and urine in the urine and liquid absorbing area. the third layer of the urine and liquid absorbing area is the core made up of wood pulp or cellulose which really is the core of the detachable urine flow collection device to absorb the urine and liquids. this provides the absorbing capacity of the detachable urine flow collection device. super absorbent polymer-sodium polyacrylate, (sap) is interwoven throughout the cellulose to allow the core to absorb urine and more liquid and maintain control of this liquid when the core is under pressure. the liberal use of sap in this layer, allows less wood pulp or cellulose to be used thus creating a more streamlined garment comprises a wood pulp or cellulose, absorbs urine and liquid, super absorbent polymer-sodium polyacrylate (sap) ul layer 3 (the core).

22. The human wearable and detachable incontinence device in accordance with claim 1, wherein said means for maintaining a waterproof layer to the fourth and final non woven layer of the urine and liquid absorbing area. this layer is a continuation of the above layer of the quick wicking area. the elements and composition of a waterproof nature of the nonwoven material used in this layer prevents urine and liquid from leaking through the urine and liquid absorbing area and wetting the user's clothes, furniture or bed linens comprises a non woven, waterproof, continuation of qw layer 4 (polyethylene nonwoven material) ul layer 4 (polyethylene nonwoven material).

23. A human wearable and detachable incontinence device for that when worn, will accept and trap urine and keep it away from contact with the user's skin until the device it is detached and disposed of, comprising:
an ellipsoid in shape, front is the widest part of the ellipse shape, back part is the narrowest part of the ellipse shape, ellipsoid shape maintained in high top to bottom, consists 3 areas, non-woven materials perform different roles in the different areas detachable urine flow collection device, for a detachable urine flow collection device that can be attached to an absorbent garment (diaper) brief or traditional underwear garment (garment) worn by a human. the detachable urine flow collection device consisting of three areas. a genital ring and opening area, a quick wicking area, and a urine and liquid absorbing area. the purpose of the detachable urine flow collection device is to direct urine and liquids away from the user to a urine and liquid absorbing area of the device. the direction and movement of urine and liquids away from the user, is attained by utilizing specific nonwoven materials in layers, and combinations, to achieve the purpose. the specific properties of the nonwoven materials and the combinations allow this movement and capturing and storage of urine and liquids to take place in the detachable urine flow collection device. the detachable urine flow collection device is designed to fit through an opening in the front portion of an disposable nonwoven garment (diaper) or brief, as well as a traditional underwear garment. the detachable urine flow collection device can be applied or removed from the attachment as needed. the genital ring and opening area of the detachable urine flow collection device assembly of the present invention mates with an opening formed in an absorbent disposable nonwoven or underwear garment. the genital ring and opening area is the top ⅓ of the detachable urine flow collection device. the connection is an intimate contact, and waterproof;
a disposable, slit opening in the front anterior panel disposable nonwoven or underwear garment, for the application of the detachable urine flow collection device. the detachable urine flow collection device fits through the garment opening in the front anterior portion of the disposable nonwoven or underwear garment. the detachable urine flow collection device attaches to the disposable nonwoven or underwear garment by velcro tape or stick type fasteners on the underside surface of the closure ring of the urine and liquid absorbing area;
a slit opening in the ant front of the disposable nonwoven or underwear garment, top inside surface opposite material velcro tape or stick type fasteners garment opening, for the application of the detachable urine flow collection device to the disposable nonwoven or underwear garment. the detachable urine flow collection device fits through the garment opening in the front anterior portion of the disposable nonwoven or underwear garment. the detachable urine flow collection device attaches to the disposable nonwoven or underwear garment by velcro tape or stick type fasteners on the underside surface of the closure ring of the urine and liquid absorbing area. these velcro tape or stick type fasteners on the underside of the closure ring attached directly to the opposite material on the inside surface of the disposable nonwoven or underwear garment opening, structurally connected to said disposable nonwoven or underwear garment, and symmetrically connected to said detachable urine flow collection device;
a top ⅓ of the detachable urine flow collection device, soft moldable nonwoven material, forms a seal around the genitals, made of nonwoven material in layers, new attribute 5 genital ring and opening area, for the genital ring and opening structurally makes up the top ⅓ of the detachable urine flow collection device and is shaped like an ellipse with the opening, made of a soft moldable nonwoven material. the front of the genital ring is the widest part of the ellipse, and moving backward it ellipses backward to its narrowest part. the closure ring is a nonwoven material that attaches to the top outside edge of the ellipsoid opening, at ninety degrees to the body of the detachable urine flow collection device. the underside of this nonwoven material closure ring is comprised of velcro tape type fasteners, or sticks tape type fasteners, or can also be one of the newer forms of elasticized nonwoven velcro tapes emerging in the absorbent garment (diaper) brief market. the tab a loose flap of nonwoven material will be attached to the font and top of the ellipsoid opening. the genital ring area is made up of the following nonwoven layers, and is described moving from the innermost layer closest to the open chamber of the detachable urine flow collection device moving outward. the innermost first layer is a through air bond (tab) nonwoven that provides separation from any wet area and the skin. the second layer is a hydrophobic nonwoven material. the third layer is a hydrophilic nonwoven material. the fourth and final layer is a polyethylene nonwoven material that is the outside layer of the detachable urine flow collection device, and is entirely hydrophobic or waterproof. the detachable urine flow collection device is designed to fit through an opening in the front portion of an absorbent disposable nonwoven garment (diaper) or brief, as well as a traditional underwear garment (garment). the function of the genital ring and opening is to accommodate the wearers genitals, furthermore the genital ring is the part of the detachable urine flow collection device that accommodates the attachment with the absorbent disposable nonwoven garment through the closure ring. the genital ring and opening of the external collection assembly of the present invention mates with the opening formed in an absorbent disposable nonwoven garment. the connection is an intimate contact, and waterproof. the underside surface of the closure ring, the fastener will make contact with the opposite nonwoven fasteners on the inside surface of the absorbent disposable non-woven garment that lines the opening of the absorbent disposable nonwoven garment. with a user wearing an absorbent disposable nonwoven garment, the widest part of the ellipsoid opening anatomically is toward the front of the user; the opening then ellipses toward the narrowest part, at the back or posterior of the user. the detachable urine flow collection device can be applied or removed from the attachment as needed by pulling on the tab and lifting the detachable urine flow collection device out of the absorbent disposable nonwoven garment. the genital ring or opening top ⅓ of the detachable urine flow collection device then progresses to the quick wicking or middle ⅓ of the detachable urine flow collection device, structurally connected to said detachable urine flow collection device;
a soft moldable, encloses and surrounds the genitals soft moldable nonwoven material, for the soft moldable nonwoven material of the genital ring and opening is designed to be in full contact and surround the users genitalia. being moldable, the material can be molded and an intimate contact can be made between the users genitals, and the detachable urine flow collection device;
a non woven material, underside surface velcro tape or stick type fasteners closure ring, for the closure ring is a nonwoven material that attaches to the underside of the genital ring and opening area it is a nonwoven with the same ellipsoid opening reflected in its form. the underside of this nonwoven material closure ring is comprised of velcro tape type fasteners, or sticks tape type fasteners, or can also be one of the newer forms of elasticized nonwoven velcro tapes emerging in the absorbent garment (diaper) brief market;
a velcro tape or stick type fasteners, follows the shape of the genital ring and opening area velcro tape or stick type fasteners, for the attachment of detachable urine flow collection device to the disposable nonwoven or underwear garment. this is achieved by velcro tape type fasteners, or sticks tape type fasteners on the underside of the nonwoven material closure ring, that adhere to a opposite material on the disposable nonwoven or underwear garment;
a non woven material tab, for assistance in the replacement of the detachable urine flow collection device. disposable nonwoven or underwear garment is pulled down to above the knees by the user. the user then removes the detachable urine flow collection device by pulling the tab this breaks the closure between the closure ring and the disposable nonwoven or underwear garment and allows the detachable urine flow collection device to be removed and a new one applied;
a non woven material, through air bonded, aids in single direction of urine and liquid flo gr layer 1 (through air bond (tab) nonwoven), for the separation of urine from the material layers of the genital ring and opening area and the users skin. this is accomplished by this innermost layer of the genital ring and opening area, being a through air bond (tab) nonwoven. the gr layer 1 (through air bond (tab) nonwoven) material allows urine and liquid to pass through it to the next layer of the genital ring and opening area but will not allow it to pass back through the layer and come in contact with the user's skin, structurally connected to said genital ring and opening area;
a non woven, hydrophobic (water molecule repellant), aids directional flow of urine and liquid gr layer 2 (hydrophobic nonwoven material), for assisting the movement of urine into and down the genital ring and opening area, and away from the skin of the user. this is accomplished by the elements and composition of a hydrophobic nonwoven material. the hydrophobic (water molecule repellant) nature of this nonwoven layer as well as gravity moves the urine and liquid toward the deeper and outermost layers of the genital ring and opening area, cohesively connected to said gr layer 1 (through air bond (tab) nonwoven), and structurally connected to said genital ring and opening area;
a non woven, hydrophilic (water molecule repellant), aids in the directional flow of urine and liquid gr layer 3 (hydrophilic nonwoven material), for moving the urine further and downward outward into the genital ring and opening area. this is accomplished by the elements and composition of a hydrophilic nature of the nonwoven material used in this layer. the hydrophillic (water molecule attractant) nature of this nonwoven layer, as well as gravity moves attracts and traps the urine and liquid toward the deeper and outermost layers of the genital ring and opening area, cohesively connected to said gr layer 2 (hydrophobic nonwoven material), and structurally connected to said genital ring and opening area;
a non woven, waterproof gr layer 4 (polyethylene nonwoven material), for maintaining a waterproof layer to the fourth and final non woven layer of the genital ring area. the elements and composition of a waterproof nature of the nonwoven material used in this layer prevents urine and liquid from leaking through the genital ring and opening area and wetting the user's clothes, furniture or bed linens, cohesively connected to said gr layer 3 (hydrophilic nonwoven material), and structurally connected to said genital ring and opening area;
a middle ⅓ of detachable urine flow collection device, ellipsoid opening, non woven layers, directs urine and liquid quick wicking area, for below the genital ring opening area is the quick wicking area middle ⅓ area of the detachable urine flow collection device. the specific purpose of the quick wicking area is to move the urine and liquid as quickly as possible to the urine and liquid collecting area. the quick wicking layer or middle ⅓ area is also ellipsoid in shape and is longer than the genital opening top ⅓ area in length from front to back. the detachable urine flow collection device is also made of layers of nonwoven material that are designed to channel urine and liquid away from the user. these layers act in a specific fashion in order to wick the urine and liquid away from the user to a specific portion of the detachable urine flow collection device. the quick wicking middle ⅓ area nonwoven layers. include the following, and is described moving from the innermost layer closest to the open chamber of the detachable urine flow collection device moving outward. the innermost first layer is a continuation of the first layer of the genital ring and is a through air bond (tab) nonwoven that provides separation from the wet area and the skin. the second layer is a continuation of the hydrophilic nonwoven material from the genital ring. this second layer is pleated and angled back toward the back and bottom of the detachable urine flow collection device to facilitate the movement of urine and liquid toward the bottom ⅓ area; the urine and liquid absorbing area. the third and final layer is a polyethylene nonwoven material that is the outside layer of the entire detachable urine flow collection device, and is entirely hydrophobic or waterproof, sequentially connected to said genital ring and opening area, and structurally connected to said detachable urine flow collection device;
a non woven, through air bonded, continuation of gr layer 1 (through air bond (tab) nonwoven) above, aids in single direction of urine and liquid flo qw layer 1 (through air bond (tab) nonwoven), for the separation of urine from the material layers of the quick wicking area and the users skin. this is accomplished by this innermost layer of the quick wicking area, being a continuation of the above innermost layer of the genital ring and is a through air bond (tab) nonwoven. the qw layer 1 (through air bond (tab) nonwoven) material allows urine and liquid to pass through it to the next layer of the quick wicking area but will not allow it to pass back through the layer and come in contact with the user's skin, structurally connected to said quick wicking area, and sequentially connected to said gr layer 1 (through air bond (tab) nonwoven);
a non woven, continuation of gr layer 3 (hydrophilic nonwoven material), aids in the directional flow of urine and liquid qw layer 2 (hydrophilic nonwoven material), for moving the urine further and downward outward into the quick wicking area. this is accomplished by this layer being a continuation of the above layer of the genital ring and opening area and the elements and composition of a hydrophilic nature of the nonwoven material used in this layer. the hydrophillic (water molecule attractant) nature of this nonwoven layer, as well as gravity moves attracts and traps the urine and liquid toward the deeper and outermost layers of the genital ring and opening area, cohesively connected to said qw layer 1 (through air bond (tab) nonwoven), structurally connected to said quick wicking area, and sequentially connected to said gr layer 3 (hydrophilic nonwoven material);
a non woven, waterproof, continuation of gr layer 4 (polyethylene nonwoven material) qw layer 3 (polyethylene nonwoven material), for maintaining a waterproof layer to the fourth and final non woven layer of the wicking area. this layer is a continuation of the above layer of the genital ring and opening area. the elements and composition of a waterproof nature of the nonwoven material used in this layer prevents urine and liquid from leaking through the quick wicking area and wetting the user's clothes, furniture or bed linens, cohesively connected to said qw layer 2 (hydrophilic nonwoven material), structurally connected to said quick wicking area, and sequentially connected to said gr layer 4 (polyethylene nonwoven material);
a bottom ⅓ of detachable urine flow collection device, ellipsoid opening, non woven layers, directs urine and liquid urine and liquid absorbing area, for the urine and liquid absorbing area, bottom ⅓ area is also ellipsoid in shape and is approximately the same size as the quick wicking layer or middle ⅓ area in length from front to back. the urine and liquid absorbing area, is made up of the following layers, and is described moving from the innermost layer closest to the open chamber of the detachable urine flow collection device moving outward. the innermost first layer is a continuation of the first layer of the quick wicking area and is a through air bond (tab) nonwoven that provides separation from the wet area and the skin. the second layer is a continuation of the hydrophillic nonwoven material from the quick wicking area. the third layer is the core made up of wood pulp or cellulose which really is the core of the detachable urine flow collection device to absorb the urine and liquids. this provides the absorbing capacity of the detachable urine flow collection device. super absorbent polymer-sodium polyacrylate, (sap) is interwoven throughout the cellulose to allow the core to absorb urine and more liquid and maintain control of this liquid when the core is under pressure. the liberal use of sap in this layer, allows less wood pulp or cellulose to be used thus creating a more streamlined garment. the fourth layer and final layer is a polyethylene non-woven material that is the outside layer of the entire detachable urine flow collection device, and is entirely hydrophobic or waterproof, sequentially connected to said quick wicking area, and structurally connected to said detachable urine flow collection device;
a non woven, through air bonded, continuation of qw layer 1 (through air bond (tab) nonwoven) above, aids in single direction of urine and liquid flo ul layer 1 (through air bond (tab) nonwoven), for the separation of urine from the material layers of the urine and liquid absorbing area and the users skin. this is accomplished by this innermost layer of the urine and liquid absorbing area, being a continuation of the above innermost layer of the quick wicking area and is a through air bond (tab) nonwoven. the qw layer 1 (through air bond (tab) nonwoven) material allows urine and liquid to pass through it to the next layer of the ui layer 1 (through air bond (tab) nonwoven), but will not allow it to pass back through the layer and come in contact with the user's skin, structurally connected to said urine and liquid absorbing area, and sequentially connected to said qw layer 1 (through air bond (tab) nonwoven);
a nonwoven, continuation of qw layer 2 (hydrophilic nonwoven material), aids in the directional flow of urine and liquid ul layer 2 (hydrophilic nonwoven material), for moving the urine further and downward outward into the urine and liquid absorbing area. this is accomplished by this layer being a continuation of the above layer of the quick wicking area and the elements and composition of a hydrophilic nature of the nonwoven material used in this layer. the hydrophillic (water molecule attractant) nature of this nonwoven layer, as well as gravity moves, attracts and traps the urine and liquid toward the deeper and outermost layers of the urine and liquid absorbing area, cohesively connected to said ul layer 1 (through air bond (tab) nonwoven), structurally connected to said urine and liquid absorbing area, and sequentially connected to said qw layer 2 (hydrophilic nonwoven material);
a wood pulp or cellulose, absorbs urine and liquid, super absorbent polymer-sodium polyacrylate (sap) ul layer 3 (the core), for the adsorption and retention of liquid and urine in the urine and liquid absorbing area. the third layer of the urine and liquid absorbing area is the core made up of wood pulp or cellulose which really is the core of the detachable urine flow collection device to absorb the urine and liquids. this provides the absorbing capacity of the detachable urine flow collection device. super absorbent polymer-sodium polyacrylate, (sap) is interwoven throughout the cellulose to allow the core to absorb urine and more liquid and maintain control of this liquid when the core is under pressure. the liberal use of sap in this layer, allows less wood pulp or cellulose to be used thus creating a more streamlined garment, cohesively connected to said ul layer 2 (hydrophilic nonwoven material), and structurally connected to said urine and liquid absorbing area; and
a non woven, waterproof, continuation of qw layer 4 (polyethylene nonwoven material) ul layer 4 (polyethylene nonwoven material), for maintaining a waterproof layer to the fourth and final non woven layer of the urine and liquid absorbing area. this layer is a continuation of the above layer of the quick wicking area. the elements and composition of a waterproof nature of the nonwoven material used in this layer prevents urine and liquid from leaking through the urine and liquid absorbing area and wetting the user's clothes, furniture or bed linens, cohesively connected to said ul layer 3 (the core), structurally connected to said urine and liquid absorbing area, and sequentially connected to said qw layer 3 (polyethylene nonwoven material).

24. The human wearable and detachable incontinence device as recited in claim 23, further comprising:
an ellipse shape, encloses the entire genital area ellipsoid Opening, for to accept and accommodate the users genitals into the detachable urine flow collection device.

25. A human wearable and detachable incontinence device for that when worn, will accept and trap urine and keep it away from contact with the user's skin until the device it is detached and disposed of, comprising:
an ellipsoid in shape, front is the widest part of the ellipse shape, back part is the narrowest part of the ellipse shape, ellipsoid shape maintained in high top to bottom, consists 3 areas, non-woven materials perform different roles in the different areas detachable urine flow collection device, for a detachable urine flow collection device that can be attached to an absorbent garment (diaper) brief or traditional underwear garment (garment) worn by a human. the detachable urine flow collection device consisting of three areas. a genital ring and opening area, a quick wicking area, and a urine and liquid absorbing area. the purpose of the detachable urine flow collection device is to direct urine and liquids away from the user to a urine and liquid absorbing area of the device. the direction and movement of urine and liquids away from the user, is attained by utilizing specific nonwoven materials in layers, and combinations, to achieve the purpose. the specific properties of the nonwoven materials and the combinations allow this movement and capturing and storage of urine and liquids to take place in the detachable urine flow collection device. the detachable urine flow collection device is designed to fit through an opening in the front portion of an disposable nonwoven garment (diaper) or brief, as well as a traditional underwear garment. the detachable urine flow collection device can be applied or removed from the attachment as needed. the genital ring and opening area of the detachable urine flow collection device assembly of the present invention mates with an opening formed in an absorbent disposable nonwoven or underwear garment. the genital ring and opening area is the top ⅓ of the detachable urine flow collection device. the connection is an intimate contact, and waterproof;
a disposable, slit opening in the front anterior panel disposable nonwoven or underwear garment, for the application of the detachable urine flow collection device. the detachable urine flow collection device fits through the garment opening in the front anterior portion of the disposable nonwoven or underwear garment. the detachable urine flow collection device attaches to the disposable nonwoven or underwear garment by velcro tape or stick type fasteners on the underside surface of the closure ring of the urine and liquid absorbing area;
a slit opening in the ant front of the disposable nonwoven or underwear garment, top inside surface opposite material velcro tape or stick type fasteners garment opening, for the application of the detachable urine flow collection device to the disposable nonwoven or underwear garment. the detachable urine flow collection device fits through the garment opening in the front anterior portion of the disposable nonwoven or underwear garment. the detachable urine flow collection device attaches to the disposable nonwoven or underwear garment by velcro tape or stick type fasteners on the underside surface of the closure ring of the urine and liquid absorbing area. these velcro tape or stick type fasteners on the underside of the closure ring attached directly to the opposite material on the inside surface of the disposable nonwoven or underwear garment opening, structurally connected to said disposable nonwoven or underwear garment, and symmetrically connected to said detachable urine flow collection device;
a top ⅓ of the detachable urine flow collection device, soft moldable nonwoven material, forms a seal around the genitals, made of nonwoven material in layers, new attribute 5 genital ring and opening area, for the genital ring and opening structurally makes up the top ⅓ of the detachable urine flow collection device and is shaped like an ellipse with the opening, made of a soft moldable nonwoven material. the front of the genital ring is the widest part of the ellipse, and moving backward it ellipses backward to its narrowest part. the closure ring is a nonwoven material that attaches to the top outside edge of the ellipsoid opening, at ninety degrees to the body of the detachable urine flow collection device. the underside of this nonwoven material closure ring is comprised of velcro tape type fasteners, or sticks tape type fasteners, or can also be one of the newer forms of elasticized nonwoven velcro tapes emerging in the absorbent garment (diaper) brief market. the tab a loose flap of nonwoven material will be attached to the font and top of the ellipsoid opening. the genital ring area is made up of the following nonwoven layers, and is described moving from the innermost layer closest to the open chamber of the detachable urine flow collection device moving outward. the innermost first layer is a through air bond (tab) nonwoven that provides separation from any wet area and the skin. the second layer is a hydrophobic nonwoven material. the third layer is a hydrophilic nonwoven material. the fourth and final layer is a polyethylene nonwoven material that is the outside layer of the detachable urine flow collection device, and is entirely hydrophobic or waterproof. the detachable urine flow collection device is designed to fit through an opening in the front portion of an absorbent disposable nonwoven garment (diaper) or brief, as well as a traditional underwear garment (garment). the function of the genital ring and opening is to accommodate the wearers genitals, furthermore the genital ring is the part of the detachable urine flow collection device that accommodates the attachment with the absorbent disposable nonwoven garment through the closure ring. the genital ring and opening of the external collection assembly of the present invention mates with the opening formed in an absorbent disposable nonwoven garment. the connection is an intimate contact, and waterproof. the underside surface of the closure ring, the fastener will make contact with the opposite nonwoven fasteners on the inside surface of the absorbent disposable non-woven garment that lines the opening of the absorbent disposable nonwoven garment. with a user wearing an absorbent disposable nonwoven garment, the widest part of the ellipsoid opening anatomically is toward the front of the user; the opening then ellipses toward the narrowest part, at the back or posterior of the user. the detachable urine flow collection device can be applied or removed from the attachment as needed by pulling on the tab and lifting the detachable urine flow collection device out of the absorbent disposable nonwoven garment. the genital ring or opening top ⅓ of the detachable urine flow collection device then progresses to the quick wicking or middle ⅓ of the detachable urine flow collection device, structurally connected to said detachable urine flow collection device;
an ellipse shape, encloses the entire genital area ellipsoid Opening, for to accept and accommodate the users genitals into the detachable urine flow collection device;
a soft moldable, encloses and surrounds the genitals soft moldable nonwoven material, for the soft moldable nonwoven material of the genital ring and opening is designed to be in full contact and surround the users genitalia. being moldable, the material can be molded and an intimate contact can be made between the users genitals, and the detachable urine flow collection device;
a non woven material, underside surface velcro tape or stick type fasteners closure ring, for the closure ring is a nonwoven material that attaches to the underside of the genital ring and opening area it is a nonwoven with the same ellipsoid opening reflected in its form. the underside of this nonwoven material closure ring is comprised of velcro tape type fasteners, or sticks tape type fasteners, or can also be one of the newer forms of elasticized nonwoven velcro tapes emerging in the absorbent garment (diaper) brief market;
a velcro tape or stick type fasteners, follows the shape of the genital ring and opening area velcro tape or stick type fasteners, for the attachment of detachable urine flow collection device to the disposable nonwoven or underwear garment. this is achieved by velcro tape type fasteners, or sticks tape type fasteners on the underside of the nonwoven material closure ring, that adhere to a opposite material on the disposable nonwoven or underwear garment;
a non woven material tab, for assistance in the replacement of the detachable urine flow collection device. disposable nonwoven or underwear garment is pulled down to above the knees by the user. the user then removes the detachable urine flow collection device by pulling the tab this breaks the closure between the closure ring and the disposable nonwoven or underwear garment and allows the detachable urine flow collection device to be removed and a new one applied;
a non woven material, through air bonded, aids in single direction of urine and liquid flo gr layer 1 (through air bond (tab) nonwoven), for the separation of urine from the material layers of the genital ring and opening area and the users skin. this is accomplished by this innermost layer of the genital ring and opening area, being a through air bond (tab) nonwoven. the gr layer 1 (through air bond (tab) nonwoven) material allows urine and liquid to pass through it to the next layer of the genital ring and opening area but will not allow it to pass back through the layer and come in contact with the user's skin, structurally connected to said genital ring and opening area;
a non woven, hydrophobic (water molecule repellant), aids directional flow of urine and liquid gr layer 2 (hydrophobic nonwoven material), for assisting the movement of urine into and down the genital ring and opening area, and away from the skin of the user. this is accomplished by the elements and composition of a hydrophobic nonwoven material. the hydrophobic (water molecule repellant) nature of this nonwoven layer as well as gravity moves the urine and liquid toward the deeper and outermost layers of the genital ring and opening area, cohesively connected to said gr layer 1 (through air bond (tab) nonwoven), and structurally connected to said genital ring and opening area;
a non woven, hydrophilic (water molecule repellant), aids in the directional flow of urine and liquid gr layer 3 (hydrophilic nonwoven material), for moving the urine further and downward outward into the genital ring and opening area. this is accomplished by the elements and composition of a hydrophilic nature of the nonwoven material used in this layer. the hydrophillic (water molecule attractant) nature of this nonwoven layer, as well as gravity moves attracts and traps the urine and liquid toward the deeper and outermost layers of the genital ring and opening area, cohesively connected to said gr layer 2 (hydrophobic nonwoven material), and structurally connected to said genital ring and opening area;
a non woven, waterproof gr layer 4 (polyethylene nonwoven material), for maintaining a waterproof layer to the fourth and final non woven layer of the genital ring area. the elements and composition of a waterproof nature of the nonwoven material used in this layer prevents urine and liquid from leaking through the genital ring and opening area and wetting the user's clothes, furniture or bed linens, cohesively connected to said gr layer 3 (hydrophilic nonwoven material), and structurally connected to said genital ring and opening area;
a middle ⅓ of detachable urine flow collection device, ellipsoid opening, non woven layers, directs urine and liquid quick wicking area, for below the genital ring opening area is the quick wicking area middle ⅓ area of the detachable urine flow collection device. the specific purpose of the quick wicking area is to move the urine and liquid as quickly as possible to the urine and liquid collecting area. the quick wicking layer or middle ⅓ area is also ellipsoid in shape and is longer than the genital opening top ⅓ area in length from front to back. the detachable urine flow collection device is also made of layers of nonwoven material that are designed to channel urine and liquid away from the user. these layers act in a specific fashion in order to wick the urine and liquid away from the user to a specific portion of the detachable urine flow collection device. the quick wicking middle ⅓ area nonwoven layers. include the following, and is described moving from the innermost layer closest to the open chamber of the detachable urine flow collection device moving outward. the innermost first layer is a continuation of the first layer of the genital ring and is a through air bond (tab) nonwoven that provides separation from the wet area and the skin. the second layer is a continuation of the hydrophilic nonwoven material from the genital ring. this second layer is pleated and angled back toward the back and bottom of the detachable urine flow collection device to facilitate the movement of urine and liquid toward the bottom ⅓ area; the urine and liquid absorbing area. the third and final layer is a polyethylene nonwoven material that is the outside layer of the entire detachable urine flow collection device, and is entirely hydrophobic or waterproof, sequentially connected to said genital ring and opening area, and structurally connected to said detachable urine flow collection device;
a non woven, through air bonded, continuation of gr layer 1 (through air bond (tab) nonwoven) above, aids in single direction of urine and liquid flo qw layer 1 (through air bond (tab) nonwoven), for the separation of urine from the material layers of the quick wicking area and the users skin. this is accomplished by this innermost layer of the quick wicking area, being a continuation of the above innermost layer of the genital ring and is a through air bond (tab) nonwoven. the qw layer 1 (through air bond (tab) nonwoven) material allows urine and liquid to pass through it to the next layer of the quick wicking area but will not allow it to pass back through the layer and come in contact with the user's skin, structurally connected to said quick wicking area, and sequentially connected to said gr layer 1 (through air bond (tab) nonwoven);
a non woven, continuation of gr layer 3 (hydrophilic nonwoven material), aids in the directional flow of urine and liquid qw layer 2 (hydrophilic nonwoven material), for moving the urine further and downward outward into the quick wicking area. this is accomplished by this layer being a continuation of the above layer of the genital ring and opening area and the elements and composition of a hydrophilic nature of the nonwoven material used in this layer. the hydrophillic (water molecule attractant) nature of this nonwoven layer, as well as gravity moves attracts and traps the urine and liquid toward the deeper and outermost layers of the genital ring and opening area, cohesively connected to said qw layer 1 (through air bond (tab) nonwoven), structurally connected to said quick wicking area, and sequentially connected to said gr layer 3 (hydrophilic nonwoven material);
a non woven, waterproof, continuation of gr layer 4 (polyethylene nonwoven material) qw layer 3 (polyethylene nonwoven material), for maintaining a waterproof layer to the fourth and final non woven layer of the wicking area. this layer is a continuation of the above layer of the genital ring and opening area. the elements and composition of a waterproof nature of the nonwoven material used in this layer prevents urine and liquid from leaking through the quick wicking area and wetting the user's clothes, furniture or bed linens, cohesively connected to said qw layer 2 (hydrophilic nonwoven material), structurally connected to said quick wicking area, and sequentially connected to said gr layer 4 (polyethylene nonwoven material);
a bottom ⅓ of detachable urine flow collection device, ellipsoid opening, non woven layers, directs urine and liquid urine and liquid absorbing area, for the urine and liquid absorbing area, bottom ⅓ area is also ellipsoid in shape and is approximately the same size as the quick wicking layer or middle ⅓ area in length from front to back. the urine and liquid absorbing area, is made up of the following layers, and is described moving from the innermost layer closest to the open chamber of the detachable urine flow collection device moving outward. the innermost first layer is a continuation of the first layer of the quick wicking area and is a through air bond (tab) nonwoven that provides separation from the wet area and the skin. the second layer is a continuation of the hydrophillic nonwoven material from the quick wicking area. the third layer is the core made up of wood pulp or cellulose which really is the core of the detachable urine flow collection device to absorb the urine and liquids. this provides the absorbing capacity of the detachable urine flow collection device. super absorbent polymer-sodium polyacrylate, (sap) is interwoven throughout the cellulose to allow the core to absorb urine and more liquid and maintain control of this liquid when the core is under pressure. the liberal use of sap in this layer, allows less wood pulp or cellulose to be used thus creating a more streamlined garment. the fourth layer and final layer is a polyethylene non-woven material that is the outside layer of the entire detachable urine flow collection device, and is entirely hydrophobic or waterproof, sequentially connected to said quick wicking area, and structurally connected to said detachable urine flow collection device;
a non woven, through air bonded, continuation of qw layer 1 (through air bond (tab) nonwoven) above, aids in single direction of urine and liquid flo ul layer 1 (through air bond (tab) nonwoven), for the separation of urine from the material layers of the urine and liquid absorbing area and the users skin. this is accomplished by this innermost layer of the urine and liquid absorbing area, being a continuation of the above innermost layer of the quick wicking area and is a through air bond (tab) nonwoven. the qw layer 1 (through air bond (tab) nonwoven) material allows urine and liquid to pass through it to the next layer of the ui layer 1 (through air bond (tab) nonwoven), but will not allow it to pass back through the layer and come in contact with the user's skin, structurally connected to said urine and liquid absorbing area, and sequentially connected to said qw layer 1 (through air bond (tab) nonwoven);
a nonwoven, continuation of qw layer 2 (hydrophilic nonwoven material), aids in the directional flow of urine and liquid ul layer 2 (hydrophilic nonwoven material), for moving the urine further and downward outward into the urine and liquid absorbing area. this is accomplished by this layer being a continuation of the above layer of the quick wicking area and the elements and composition of a hydrophilic nature of the nonwoven material used in this layer. the hydrophillic (water molecule attractant) nature of this nonwoven layer, as well as gravity moves, attracts and traps the urine and liquid toward the deeper and outermost layers of the urine and liquid absorbing area, cohesively connected to said ul layer 1 (through air bond (tab) nonwoven), structurally connected to said urine and liquid absorbing area, and sequentially connected to said qw layer 2 (hydrophilic nonwoven material);
a wood pulp or cellulose, absorbs urine and liquid, super absorbent polymer-sodium polyacrylate (sap) ul layer 3 (the core), for the adsorption and retention of liquid and urine in the urine and liquid absorbing area. the third layer of the urine and liquid absorbing area is the core made up of wood pulp or cellulose which really is the core of the detachable urine flow collection device to absorb the urine and liquids. this provides the absorbing capacity of the detachable urine flow collection device. super absorbent polymer-sodium polyacrylate, (sap) is interwoven throughout the cellulose to allow the core to absorb urine and more liquid and maintain control of this liquid when the core is under pressure. the liberal use of sap in this layer, allows less wood pulp or cellulose to be used thus creating a more streamlined garment, cohesively connected to said ul layer 2 (hydrophilic nonwoven material), and structurally connected to said urine and liquid absorbing area; and
a non woven, waterproof, continuation of qw layer 4 (polyethylene nonwoven material) ul layer 4 (polyethylene nonwoven material), for maintaining a waterproof layer to the fourth and final non woven layer of the urine and liquid absorbing area. this layer is a continuation of the above layer of the quick wicking area. the elements and composition of a waterproof nature of the nonwoven material used in this layer prevents urine and liquid from leaking through the urine and liquid absorbing area and wetting the user's clothes, furniture or bed linens, cohesively connected to said ul layer 3 (the core), structurally connected to said urine and liquid absorbing area, and sequentially connected to said qw layer 3 (polyethylene nonwoven material).
